SEQUENCE LISTING (27 sequences)

SEQ ID NO: 1 - DNA, 3121 bp, Artificial sequence
  Interleukin-1 receptor antagonist (IL-1Ra) splice variant
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaaggtgagtggttgccaggaaagccaatgtatgtgggcatcacgtcactttgcccgtctgtctgcagcagcatggcctgcctgcacaaaccctaggtgcaatgtcctaatccttgttgggtctttgtattcaagtttgaagctgggagggcctggctactgaagggcacatatgagggcagcctgaagagggtgtggagaggtagagtctaggtcagaggtcagtgcctataggcacagtggtcccagggccacagctgggaagggcaaataccagaaggcaaggttgaccattcccttcctcaagtgcctattaaggctccatgttcctatgttgttcaaaccctaactcaatcccaaattaatccaccatgtataaggttgagctatgtctcttattcctggacaccatactcagccatattctggtccacacattaaacaagctggatgaccttgaagaagcttcacccactctgttcctcagctttcccttcagtgggatgatatcaactggacaacaggatgtgcgattcttttagttccagccttccaggatgttttcactcccctgtttgttgttgtaggatggtattacctccaccttcccaccttccctatgccctggttctgtctcctgtgcctcgctctgaaagtggatgagacctacaattcctgtcctggtagttctcctaatgaacacactgaagcacgaggaagctgagatttttgttgctacatgagagcatggaggcctcttagggagagaggaggttcagagactcctaggctcctgtggagccccactcatggccttgttcattttccctgcccctcagcaacactcctattgacctggagcacaggtatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcttggtttaatcttctatttacctgcagaccaggaagatgagacctctctgcccttctgacctcgggattttagttttgtggggaccaggggagatagaaaaatacccggggtctcttcattattgctgcttcctcttctattaacctgaccctcccctctgttcttccccagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcc

SEQ ID NO: 2 - protein, 98 aa, Artificial sequence
  IL-1Ra splice variant
MEICRGLRSHLITLLLFLFHSETICRPSGRKSSKMQAFRIWDVNQKTFYLRNNQLVAGYLQGPNVNLEGEWLPGKPMYVGITSLCPSVCSSMACLHKP

SEQ ID NO: 3 - DNA, 2991 bp, Artificial sequence
  IL-1Ra splice variant
acgatctgccgaccctctgggagaaaatccagcaagatgcaagccttcagaatctgggatgttaaccagaagaccttctatctgaggaacaaccaactagttgctggatacttgcaaggaccaaatgtcaatttagaaggtgagtggttgccaggaaagccaatgtatgtgggcatcacgtcactttgcccgtctgtctgcagcagcatggcctgcctgcacaaaccctaggtgcaatgtcctaatccttgttgggtctttgtattcaagtttgaagctgggagggcctggctactgaagggcacatatgagggcagcctgaagagggtgtggagaggtagagtctaggtcagaggtcagtgcctataggcacagtggtcccagggccacagctgggaagggcaaataccagaaggcaaggttgaccattcccttcctcaagtgcctattaaggctccatgttcctatgttgttcaaaccctaactcaatcccaaattaatccaccatgtataaggttgagctatgtctcttattcctggacaccatactcagccatattctggtccacacattaaacaagctggatgaccttgaagaagcttcacccactctgttcctcagctttcccttcagtgggatgatatcaactggacaacaggatgtgcgattcttttagttccagccttccaggatgttttcactcccctgtttgttgttgtaggatggtattacctccaccttcccaccttccctatgccctggttctgtctcctgtgcctcgctctgaaagtggatgagacctacaattcctgtcctggtagttctcctaatgaacacactgaagcacgaggaagctgagatttttgttgctacatgagagcatggaggcctcttagggagagaggaggttcagagactcctaggctcctgtggagccccactcatggccttgttcattttccctgcccctcagcaacactcctattgacctggagcacaggtatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcttggtttaatcttctatttacctgcagaccaggaagatgagacctctctgcccttctgacctcgggattttagttttgtggggaccaggggagatagaaaaatacccggggtctcttcattattgctgcttcctcttctattaacctgaccctcccctctgttcttccccagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcc

SEQ ID NO: 4 - protein, 76 aa, Artificial sequence
  IL-1Ra splice variant
TICRPSGRKSSKMQAFRIWDVNQKTFYLRNNQLVAGYLQGPNVNLEGEWLPGKPMYVGITSLCPSVCSSMACLHKP

SEQ ID NO: 5 - DNA, 297 bp, Artificial sequence
  IL-1Ra splice variant - alternative codon usage
atggagatttgtcgtggtttgaggtcacatttgattacattgttgttgtttttatttcacagcgaaactatttgtcgtcctagcggacgtaagagctctaaaatgcaggcttttcgtatttgggacgtcaatcaaaaaacattttacttacgtaataatcagttggtcgcaggctatctccagggtcctaacgtgaacctcgagggtgagtggttgccaggaaagccaatgtatgtgggcatcacgtcactttgcccgtctgtctgcagcagcatggcctgcctgcacaaaccctag

SEQ ID NO: 6 - DNA, 231 bp, Artificial sequence
  IL-1Ra splice variant - alternative codon usage
actatttgtcgtcctagcggacgtaagagctctaaaatgcaggcttttcgtatttgggacgtcaatcaaaaaacattttacttacgtaataatcagttggtcgcaggctatctccagggtcctaacgtgaacctcgagggtgagtggttgccaggaaagccaatgtatgtgggcatcacgtcactttgcccgtctgtctgcagcagcatggcctgcctgcacaaaccctag

SEQ ID NO: 7 - DNA, 1740 bp, Homo sapiens
gaattccgggctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccaggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgccaaaaaaaaaaaaaaaaaaaccggaatcccggccggaattc

SEQ ID NO: 8 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 9 - protein, 180 aa, Homo sapiens
MALADLYEEGGGGGGEGEDNADSKETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 10 - protein, 143 aa, Homo sapiens
MQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 11 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 12 - DNA, 578 bp, Homo sapiens
cagaagacctcctgtcctatgaggccctccccatggctttagctgacttgtatgaagaaggaggtggaggaggaggagaaggtgaagacaatgctgactcaaaggagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtac

SEQ ID NO: 13 - DNA, 777 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacaagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 14 - DNA, 1802 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 15 - DNA, 20 bp, Artificial sequence
  PCR primer
cagaggcctccgcagtcacc

SEQ ID NO: 16 - DNA, 20 bp, Artificial sequence
  PCR primer
tgacgggctggtcagcttcc

SEQ ID NO: 17 - DNA, 21 bp, Artificial sequence
  PCR primer
ggcagcctgaagagggtgtgg

SEQ ID NO: 18 - DNA, 23 bp, Artificial sequence
  PCR primer
tcccactgaagggaaagctgagg

SEQ ID NO: 19 - DNA, 54 bp, Artificial sequence
  PCR primer
ggggacaagtttgtacaaaaaagcaggctccatggaaatctgcagaggcctccg

SEQ ID NO: 20 - DNA, 54 bp, Artificial sequence
  PCR primer
ggggacaagtttgtacaaaaaagcaggctccatgacgatctgccgaccctctgg

SEQ ID NO: 21 - DNA, 57 bp, Artificial sequence
  PCR primer
ggggaccactttgtacaagaaagctgggtagacccaacaaggattaggacattgcac

SEQ ID NO: 22 - DNA, 51 bp, Artificial sequence
  PCR primer
ggggaccactttgtacaagaaagctgggtatccctgcagtccttgccatgc

SEQ ID NO: 23 - DNA, 29 bp, Artificial sequence
  PCR primer
ggggacaagtttgtacaaaaaagcaggct

SEQ ID NO: 24 - DNA, 28 bp, Artificial sequence
  PCR primer
ggggaccactttgtacaagaaagctggg

SEQ ID NO: 25 - DNA, 21 bp, Artificial sequence
  PCR primer
cgcgttaacgctagcatggat

SEQ ID NO: 26 - DNA, 23 bp, Artificial sequence
  PCR primer
cacagagttttagagactacaat

SEQ ID NO: 27 - protein, 17 aa, Artificial Sequence
  peptide used for immunization
GEWLPGKPMYVGITSLC

(19) United States
(12) Patent Application Publication
(10) Pub. No.: US 2005/0159590 A1
(43) Pub. Date: Jul. 21, 2005

(54) Variants of interleukin-1 receptor antagonist: compositions and uses thereof

(76) Inventors: Galit Rotman, Herzlia (IL); Dalit Milo-Landesman, Givatayim (IL); Dvir Dahary, Tel Aviv (IL); Ronen Shemesh, Modi'in (IL); Jeanne Bernstein, Kfar Yona (IL); Dani Eshel, Pardes-Hana Karkur (IL)

Correspondence Address: WINSTON & STRAWN LLP, 1700 K STREET, N.W., WASHINGTON, DC, 20006, US

(21) Appl. No.: 10/926,773

(22) Filed: Aug. 25, 2004

Related U.S. Application Data
(60) Provisional application No. 60/497,324, filed on Aug. 25, 2003.

(51) Int. Cl. C12Q 1/68; C07H 21/04; A61K 38/20; C07K 14/52; C07K 16/28; A61K 48/00

(52) U.S. Cl. 530/351; 530/388.22; 536/23.5; 435/69.52; 435/320.1; 435/325; 435/6; 514/44; 424/85.2

(57) ABSTRACT

The present invention provides Interleukin-1 receptor antagonist splice variants, including isolated nucleic acids encoding these variants and the encoded amino acid sequences, as well as antibodies, antisense oligonucleotides, expression vectors and host cells that include these sequences. The present invention further discloses methods of using these sequences in the diagnosis, prognosis, treatment, and prevention of diseases and disorders mediated by Interleukin-1.

This application claims the benefit of U.S. Provisional Application No. 60/497,324 filed Aug. 25, 2003, the entire content of which is expressly incorporated herein by reference thereto.

### FIELD OF THE INVENTION

The present invention relates to nucleic acid and amino acid sequences of an Interleukin-1 receptor antagonist splice variant and to the use of these sequences in the diagnosis, prognosis, treatment, and prevention of diseases and disorders mediated by Interleukin-1.

### BACKGROUND OF THE INVENTION

Cytokines, such as Interleukin-1 (IL-1), are an important part of the response of an organism to infection and injury. IL-1 includes a class of proteins produced by numerous cell-types, including monocytes and some macrophages. This class includes at least two 17-18 kilodalton proteins known as IL-1α and IL-1β, which are agonists for IL-1 receptor (IL-1R). These proteins have important physiological effects on a number of different target cells involved in the inflammatory and immune responses. The proteins are co-mitogens (with phytohemaglutinin) for T-cells, cause both fibroblasts and chondrocytes to secrete latent collagenase, and increase the surface adhesive powers of endothelial cells for neutrophils. In addition, they act on the hypothalamus as pyrogens, they stimulate the catabolism of muscle protein, and they cause hepatocytes to synthesize a class of proteins known as “acute phase reactants.” Acute phase reactants are produced by the liver in higher levels during the acute part of the response to inflammation or infection.

There are two specific immunoglobulin-like membrane bound IL-1R variants: type I and type II. Type I is expressed at low concentration in T cells, endothelial cells and fibroblasts; it has a long cytoplasmic tail and is capable of inducing intracellular signaling. Type II, on the other hand, is expressed on B cells, monocytes and neutrophiles, has a short cytoplasmic tail and is not functionally active. It is believed to act as a decoy receptor (soluble receptor through shedding or alternative splicing). Unless otherwise noted herein, the terms “IL-1R” or “IL-1 receptor” refer to the type I, physiologically active receptor.

Because IL-1 is involved in the body's response to inflammation, it is not surprising that excessive production or activity of IL-1 can lead to inflammatory diseases. Such pathological processes, commonly referred to as “interleukin-1 mediated diseases”, are associated with elevated levels of IL-1 in bodily fluids or tissues. There is a large body of evidence currently available which supports the role of IL-1 as a major mediator of the systemic response to diseases and as an activator of the remaining members of the cytokine cascade (Dinarello C A 1994 FASEB J 8: 1314-1325). A non-exclusive list of acute and chronic interleukin-1 (IL-1)-mediated inflammatory diseases includes but is not limited to the following: autoimmune diseases; acute pancreatitis; ALS (Amyotrophic Lateral Sclerosis, also known as Lou Gehrig's Disease); Alzheimer's disease; cachexia/anorexia; asthma; atherosclerosis; chronic fatigue syndrome, fever; diabetes (e.g., insulin-dependent diabetes); glomerulonephritis; graft versus host rejection; hemorrhagic shock; hyperalgesia, inflammatory bowel disease; inflammatory conditions of a joint including osteoarthritis, psoriatic arthritis and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS (adult respiratory distress syndrome)); multiple myeloma; multiple sclerosis; myelogenous leukemia (e.g., AML (Acute Myelogenous Leukemia) and CML (Chronic Myelogenous Leukemia)) and other leukemias; myopathies (e.g., muscle protein metabolism, especially in sepsis); osteoporosis; Parkinson's disease; chronic pain; pre-term labor; psoriasis; reperfusion injury; septic shock; side effects from radiation therapy, temporal mandibular joint disease, tumor metastasis; or an inflammatory condition resulting from strain, sprain, cartilage damage, trauma, orthopedic surgery, infection or other disease processes.

U.S. Pat. No. 6,599,873, hereby incorporated by reference as if fully set forth herein, describes a number of pathological conditions associated with IL-1 production. For example, without wishing to be limited by a single hypothesis, IL-1 may increase the level of collagenase in an arthritic joint, as well as being potentially involved in immunopathology of rheumatoid arthritis. IL-1 may alter endothelial cell function and thereby cause the migration of leukocytes and lymphocytes into the synovial tissue, as well as causing macrophages to accumulate in the synovial lining. In addition, IL-1 may cause capillary growth and vascularization. IL-1 may also be at least partially responsible for tissue damage in rheumatoid arthritis, by stimulating release of enzymes from fibroblasts and chondrocytes.

IL-1 may also be associated with damage and/or pathological functioning in various types of arthritis. Excessive IL-1 production has been demonstrated in the skin of patients with psoriasis and high levels of IL-1 can be found in the synovial fluid of patients with psoriatic arthritis. IL-1 released by cells in the inflamed synovium in psoriatic arthritis may mediate tissue destruction through stimulation of enzyme release from other cells. The joint pathology of Reiter's syndrome is similar to that seen in psoriatic arthritis and in rheumatoid arthritis. IL-1 has been implicated as a mediator of tissue destruction in these three different forms of inflammatory arthritis. Moreover, IL-1 maybe found in the synovial fluid of patients with osteoarthritis. The release of IL-1 by chondrocytes has been implicated in the destruction of articular cartilage in this disease.

IL-1 may also increase the severity of autoimmune diseases. For example, altered IL-1 production has been described from peripheral blood cells in persons suffering from systemic lupus erythematosus. Moreover, some of the alterations in B lymphocyte function may be related to abnormalities in IL-1 production or IL-1 availability.

Excessive IL-1 production has been demonstrated in the peripheral monocytes of patients with scleroderma, and IL-1 has been implicated as a possible agent of fibrosis through stimulation of collagen production by fibroblasts. The mechanism of tissue damage in dermatomyositis might also involve cell-mediated immunity and IL-1 may therefore be involved as a mediator in this pathophysiological process.

Acute and chronic interstitial lung disease is characterized by excessive collagen production by lung fibroblasts which may be stimulated by IL-1. Recent studies on an animal model of pulmonary hypertension indicate that IL-1 may be responsible for induction of endothelial cell changes that result in narrowing of pulmonary arteries. It is this narrowing that leads to pulmonary hypertension and further secondary damage. Thus, IL-1 inhibitors could be useful in treating these lung diseases.

Recent studies have described that IL-1 is capable of directly damaging the beta cells in the Islets of Langerhans that are responsible for the production of insulin. IL-1 damage to the cells is now hypothesized to be a primary event in the acute phase of juvenile diabetes mellitus.

Monocyte and macrophage infiltration in the kidneys predominates in many forms of acute and chronic glomerulonephritis. IL-1 release by these cells may result in local accumulation of other inflammatory cells, eventually leading to inflammatory damage and fibrotic reaction in the kidneys.

It has been demonstrated that the crystals found in tissues or fluids in gout or pseudogout can directly stimulate macrophages to release IL-1. Thus, IL-1 may be an important mediator in the inflammatory cycle in these diseases.

IL-1 is one of the important endogenous pyrogens and may be responsible for inducing the marked degree of fever seen in some infectious diseases such as acute febrile illnesses due to bacteria or viruses.

Sarcoidosis is characterized by granulomatous lesions in many different organs in the body. IL-1 has been shown to be capable of inducing granuloma formation in vitro and may be involved in this process in patients with sarcoidosis.

Excessive IL-1 production has been demonstrated in peripheral monocytes from both Crohn's disease and ulcerative colitis. Local IL-1 release in the intestine may be an important mediator in stimulating the inflammatory cycle in these diseases.

Certain lymphomas are characterized by fever, osteoporosis and even secondary arthritis. Excessive IL-1 release has been demonstrated by some lymphoma cells in vitro and may be responsible for some of the clinical manifestations of these malignancies. Also, IL-1 production by some malignant lymphocytes may be responsible for some of the fever, acute phase response and cachexia seen with leukemias.

IL-1 release by astrocytes in the brain is thought to be responsible for inducing the fibrosis that may result after damage to the brain from vascular occlusion.

In these and other circumstances in which IL-1 has a harmful effect, compounds which inhibit IL-1 activity clearly have a pharmaceutical use. Thus, systemically administered, IL-1 inhibitors could be useful immunosuppressive agents. Locally applied, such IL-1 inhibitors could serve to prevent tissue destruction in inflamed joints and other sites of inflammation.

Interleukin-1 receptor antagonist (IL-1Ra) is a human protein that acts as a natural inhibitor of interleukin-1, by binding to the receptor (IL-1R) but without inducing signaling, and hence without inducing the physiological effects of IL-1 agonist ligands. IL-1Ra also does not allow docking of IL-1R accessory proteins. IL-1Ra is structurally related to IL-1 alpha and IL-1 beta, sharing 30% and 19% amino acid sequence homology with IL-1 alpha and IL-1 beta respectively. IL-1R type I binds IL-1Ra more avidly than IL-1 beta; IL-1R type II binds IL-1 beta more avidly than IL-1Ra.

The IL-1Ra protein was first isolated and purified from monocytes, and the amino acid sequence and the polynucleotide sequence encoding same were disclosed (U.S. Pat. No. 5,075,222, hereby incorporated by reference as if fully set forth herein). Additional DNA sequences and proteins identified as inhibitors of interleukin-1 receptor disclosed, for example, in published PCT Application Nos WO 96/09323; WO 96/12022, WO 00/20595, and in U.S. Pat. Nos. 5,455,330; 5,874,561 and 6,541,623, all of which are hereby incorporated by reference as if fully set forth herein. The GenBank references of known IL-1Ra variants are: gi:32576 (mRNA) and gi:32577 (amino acid) for the wild type; gi:27894315 (mRNA) and gi: 10835147 (amino acid) for the known secreted splice variant; gi:2997620 and gi: 1008970 (mRNAs) and their respective amino acid references gi:2997621 and gi:1008971 for the two known intracellular variants. The secreted splice variant is secreted by activated monocytes, neutrophils, macrophages, synovial and dermal fibroblasts, and other types of cells.

In normal homeostasis, the actions of IL-1 are maintained in balance by IL-1Ra, other natural IL-1 inhibitors (IL-1RII, circulating soluble IL-1 RI and IL-1RII) and a network of anti-inflammatory cytokines. However, in rheumatoid arthritis, an imbalance exists in which IL-1 is present in the synovial fluid at a rate 9 times higher than IL-1Ra. This imbalance favors agonist-derived inflammation and destruction.

These findings have also been supported in animal models. For example, IL-1Ra deficient mice spontaneously develop autoimmune diseases similar to R.A (rheumatoid arthritis) and arthritis. Immune colitis in rabbits depends on production of IL-1 and is ameliorated by exogenous administration of IL-1Ra.

Therefore, clinical implications for an imbalance between IL-1 and IL-1Ra include but are not limited to, rheumatoid arthritis, asthma, inflammatory bowel disease, transplant rejection, and bone marrow transplantation. It is also believed that this imbalance may be implicated in cancers such as leukemias and myelomas, and possibly also in arteriosclerosis, Alzheimer's disease and septic shock.

Unfortunately, IL-1Ra itself has a number of drawbacks as a therapeutic molecule. For example, a large excess of IL-1Ra is required to block the effect of IL-1. The antagonist has a short (6 hours) half-life in blood plasma. Also, daily injections are required to sustain a therapeutic effect.

U.S. Pat. Nos. 5,747,444 and 5,817,306 describe a method for treating graft versus host disease by administering a recombinant IL-Ra; although such treatment may be effective from the point of view of the biological mechanism, as noted above such treatment has many practical barriers to actual clinical efficacy.

U.S. Pat. No. 5,872,095 discloses a method for reducing reperfusion injury, as well as methods for inhibiting IL-1 induced expression of a leukocyte adhesion molecule by endothelial cells, treating disease states resulting from IL-1 induced adhesion of leukocytes to endothelial cells, and treating arthritis, all by administering a specific type of IL-1Ra variant.

U.S. Pat. No. 6,159,460 describes the use of the wild type (WT) IL-1 receptor antagonist for treatment of reperfusion injury. Thus, different types of IL-1Ra variants may be expected to be useful for the treatment of reperfusion injury.

U.S. Pat. No. 6,027,712 describes localized treatment of inflamed mucosal tissue lining a cavity with the ear, nose or sinus with IL-1Ra using a special formulation of aerosol.

U.S. Pat. No. 5,747,072 describes a method of reducing an inflammatory response in a joint by administering to the joint a recombinant adenoviral vector comprising an expression control sequence operatively linked to a gene that encodes an IL-1 receptor antagonist, and expressing said IL-1Ra at a level sufficient to reduce an inflammatory response in said joint. U.S. Pat. No. 6,096,728 provides pharmaceutical compositions comprising synergistic amounts of a hyaluronan or a salt thereof, and an IL-1Ra.

The level of IL-1 receptor antagonist within a cell or a tissue may also have diagnostic value, for example by diagnosing endometrial cancer by measuring the amount of intracellular IL-1Ra present in endometrial cells from a patient suspected of having said cancer, and comparing the amount to that present in normal endothelial cells is disclosed in U.S. Pat. No. 5,840,496. Methods for diagnosing diseases resulting from undesirable cell adhesion of IL-1 receptor positive cells to biological material, particular to endothelial cells, or autoimmune related diseases, or IL-1 dependent cancer by measuring the amount of intracellular IL-1Ra present are disclosed in U.S. Pat. No. 5,814,469.

As described herein above, interleukin-1 is involved in many pathological conditions, and since IL-1Ra itself is deficient as a therapeutic protein, various modes of inactivation of IL-1, together with advanced methods of applications thereof are therefore required for the treatment of different IL-1 mediated diseases. However, the background art does not teach all naturally occurring splice variants of interleukin-1 receptor antagonists.

### SUMMARY OF THE INVENTION

The present invention overcomes deficiencies of the background art by providing novel Interleukin-1 receptor antagonist (IL-1Ra) splice variants, referred to herein after as CGEN-R1. According to one embodiment the novel splice variants are mammalian; a preferred embodiment being a human IL-1Ra splice variant. The present invention provides isolated polynucleotides encoding the novel splice variants, including recombinant DNA molecules. The present invention also provides antibodies that specifically recognize one or more epitopes present on such splice variants, capable of distinguishing the novel variants from previously known interleukin-1 receptor antagonists.

The present invention further provides vectors and host cells, including expression vectors containing the polynucleotides of the invention, cells engineered to contain the polynucleotides of the present invention, cells genetically engineered to express the polynucleotides of the present invention, and methods of using same for producing recombinant IL-1Ra splice variants according to the present invention.

These novel IL-1Ra splice variants or polynucleotides encoding same can be prepared in a pharmaceutical composition. Also, the present invention to provides methods for the diagnosis and treatment of IL-1 related diseases comprising administering to a subject in need thereof a pharmaceutical composition comprising as an active ingredient selected from CGEN-R1, variants thereof and polynucleotides encoding same.

Without wishing to be limited by a single hypothesis, the CGEN-R1 variant according to the present invention can act as a “decoy” for IL-1 (the ligand of the IL-1 receptor), and therefore prevent the ligand from inducing its physiological activities by blocking signal transduction through binding to the receptor.

According to one aspect, the present invention provides IL-1Ra splice variant polypeptides, peptides derived therefrom, and polynucleotides encoding same.

According to one embodiment, the present invention provides an isolated polynucleotide comprising a genomic, complementary or composite polynucleotide sequence encoding a polypeptide capable of binding to a mammalian IL-1 receptor which is at least 80%, preferably at least 85%, more preferably at least 90% or more, and most preferably at least 95% or 100% homologous (similar+identical amino acids) to CGEN-R1 including signal peptide (SEQ ID NO:2):

| | |
|---|---|
| MEICRGLRSH LITLLLFLFH SETICRPSGR KSSKMQAFRI WDVNQKTFYL RNNQLVAGYL | 60 |
| QGPNVNLEGE WLPGKPMYVG ITSLCPSVCS SMACLHKP | 98 |

or CGEN-R1 without signal peptide (SEQ ID NO:4):

| | |
|---|---|
| RPSGRKSSKM QAFRIWDVNQ KTFYLRNNQL VAGYLQGPNV NLEGEWLPGK PMYVGITSLC | 60 |
| PSVCSSMACL HKP | 73 |

Preferably, the encoded polypeptide is substantially devoid of at least one, and more preferably all, IL-1 activity.

The degree of homology may be determined using appropriate alignment software as is known in the art.

According to one preferred embodiment, the polynucleotide according to the present invention encodes a polypeptide comprising contiguous amino acids having at least 80%, preferably at least 90%, more preferably 95% or more homology (similar+identical amino acids) to positions 44 to 73 of SEQ ID NO:4.

According to another preferred embodiment, the polypeptide of the present invention is of human origin.

According to another embodiment, the present invention provides polynucleotides encoding for the IL-1Ra splice variants, including an isolated polynucleotide encoding a polypeptide comprising the sequence of SEQ ID NO:2 or SEQ ID NO:4 or fragments, variants and analogs thereof. The invention also provides a polynucleotide sequence which hybridizes under stringent conditions to the polynucleotide sequence encoding the amino acid sequence of SEQ ID NO:2 or 4, or fragments of these polynucleotide sequences. The invention further provides a polynucleotide sequence comprising the complement of the polynucleotide sequence encoding the amino acid sequence of SEQ ID NO:2 or 4, or fragments or variants of said polynucleotide sequence.

According to another embodiment, the isolated polynucleotides of the present invention further includes a polynucleotide comprising a nucleotide sequence selected from the group consisting of SEQ ID NOs:1, 3, 5, or 6 (FIG. 2), or fragments, variants and analogs thereof. The present invention further provides the complement sequence for a polynucleotide sequence selected from the group consisting of SEQ ID NOs:1, 3, 5 or 6, or fragments, variants and analogs thereof. The polynucleotides of the present invention also include a polynucleotide that hybridizes to the complement of the nucleotide sequence selected from the group consisting of SEQ ID NOs:1, 3, 5, and 6 under stringent hybridization conditions.

According to another embodiment, the present invention provides a polypeptide comprising an amino acid sequence selected from the group consisting of SEQ ID NO:2, SEQ ID NO:4 and fragments, variants and analogs thereof, wherein the polypeptide binds to a mammalian IL-1 receptor. Preferably, the polypeptide is substantially devoid of IL-1 activity.

According to one embodiment, the present invention provides a polypeptide having an amino acid sequence which is at least 80%, preferably at least 85%, more preferably at least 90%, and most preferably at least 95% homologous (similar+identical amino acids) to SEQ ID NO:2 or 4.

According to yet another embodiment, the polypeptide comprises contiguous amino acids having at least 80%, preferably at least 90%, more preferably 95% or more homology (similar+identical amino acids) to positions 44 to 73 of SEQ ID NO:4.

According to yet another embodiment, the present invention provides an expression vector containing at least a fragment of any of the polynucleotide sequences having SEQ ID NOs:1, 3, 5, or 6 (FIG. 2). In yet another embodiment, the expression vector containing the polynucleotide sequence is contained within a host cell. The present invention further provides a method for producing the polypeptides according to the present invention comprising a) culturing a host cell containing an expression vector containing at least a fragment of the polynucleotide sequence encoding CGEN-R1 under conditions suitable for the expression of the polypeptide; and b) recovering the polypeptide from the host cell culture.

According to another aspect the present invention also provides a method for detecting a polynucleotide which encodes IL-Ra in a biological sample comprising the steps of: a) hybridizing the complement of the polynucleotide sequence which encodes SEQ ID NO:2 or 4 to nucleic acid material of a biological sample, thereby forming a hybridization complex; and b) detecting the hybridization complex, wherein the presence of the complex correlates with the presence of a polynucleotide encoding IL-1Ra in the biological sample. According to one embodiment the nucleic acid material of the biological sample is amplified by a polymerase chain reaction prior to hybridization.

According to yet another aspect the present invention provides a pharmaceutical composition comprising a polypeptide having the amino acid sequence of SEQ ID NO:2 or 4 or a polynucleotide encoding same, further comprising a pharmaceutically acceptable diluent or carrier.

According to a further aspect the present invention provides a purified antagonist of the IL-1Ra splice variant of the present invention. The antagonist, specifically an antibody, has a number of applications, including identification, purification and detection of IL-1Ra, specifically CGEN-R1.

According to one embodiment, the present invention provides a purified antibody which binds to at least one epitope of a polypeptide comprising the amino acid sequence of SEQ ID NO:2 or 4 or fragments, analogs and variants thereof.

According to preferred embodiments of the present invention, there is provided a CGEN-R1 protein, comprising a first portion having an amino acid sequence being at least about 90% homologous, and preferably at least about 95% homologous, to amino acids 1-68 of wild type IL-1Ra (SEQ ID NO:8, which is GenBank record gi:32577; FIG. 1A), which are also amino acids 1-68 of SEQ ID NO:2; and a second portion having an amino acid sequence being at least about 80%, preferably at least about 85%, more preferably at least about 90% and most preferably at least about 95% homologous to an amino acid sequence GEWLPGKPMYVGITSLCPSVCSSMACLHKP (amino acids 69-98 of SEQ ID NO:2), wherein said first and second portions are contiguous and in sequential order.

According to other preferred embodiments of the present invention, there is provided a tail portion of CGEN-R1 according to the present invention, which comprises a peptide according to an amino acid sequence GEWLPGKPMYVGITSLCPSVCSSMACLHKP, or a sequence at least about 80%, preferably at least about 85%, more preferably at least about 90% and most preferably at least about 95% homologous to this amino acid sequence. This peptide is present at amino acids 69-98 of the CGEN-R1 sequence comprising a signal peptide according to the present invention (SEQ ID NO:2).

According to another aspect, the novel splice variants as disclosed in the present invention comprise a unique sequence in the region joining or bridging the novel tail sequences comprising amino acids 69-98 of SEQ ID NO:2 and amino acids 44-73 of SEQ ID NO:4 to the known subsequence comprising amino acids 1-68 that is common to CGEN-R1 as set forth in SEQ ID NO:2 and the previously known IL-1Ra as set forth in SEQ ID NO:8. The unique joining or bridging region is a feature that characterizes the novel splice variants according to the present invention and distinguishes them from the previously known variants. This bridge portion may also comprise an epitope that is specific to the novel splice variants of the invention.

According to additional preferred embodiments of the present invention, there is provided a bridge portion of SEQ ID NO:2, comprising a peptide sequence having a length “n”, wherein n is from about 4 to 50 amino acids, preferably from about 5 to 40 amino acids, more preferably 6-30 amino acids, the bridge portion comprising at least the dipeptide EG at positions 68-69 of SEQ ID NO:2 (marked with underlining on FIG. 1A), the bridge portion defined as follows (following the numbering set forth in SEQ ID NO:2): a sequence starting from any of amino acid numbers 68−x to 68; and ending at any of amino acid numbers 69+((n−2)−x), in which x varies from 0 to n−2; wherein the total amino acid number in the bridge portion does not exceed 98.

According to other preferred embodiments, the bridge portion above optionally comprises a polypeptide being at least about 80%, preferably at least about 85%, more preferably at least about 90% and most preferably at least about 95% homologous to at least one sequence described above.

Similarly, the bridge portion can optionally be relatively short, such as from about 4 to about 9 amino acids in length. For four amino acids, the first bridge portion would comprise the following peptides: EGEW; NLEG; LEGE. All peptides feature EG as a portion thereof. Peptides of from about five to about nine amino acids could optionally be similarly constructed.

According to yet further aspects, the present invention provides methods for preventing, treating or ameliorating an IL-1 related disease or disorder, comprising administering to a subject in need thereof a pharmaceutical composition comprising as an active ingredient an IL-1Ra splice variant.

According to one embodiment, the present invention provides a method for preventing, treating or ameliorating an IL-1 related diseases or disorder, comprising administering to a subject in need thereof a pharmaceutical composition comprising as an active ingredient a polypeptide comprising the SEQ ID NO:2 or SEQ ID NO:4 or fragments, variants and analogs thereof.

According to another embodiment, the present invention provides a method for preventing, treating or ameliorating an IL-1 related disease or disorder, comprising administering to a subject in need thereof a pharmaceutical composition comprising as an active ingredient a polynucleotide encoding a polypeptide comprising the SEQ ID NO:2 or SEQ ID NO:4 or fragments, variants and analogs thereof.

According to yet another embodiment, the IL-1 related disease or disorder is selected from the group consisting of acute pancreatitis; ALS; Alzheimer's disease; cachexia/anorexia; asthma; atherosclerosis; chronic fatigue syndrome; diabetes (e.g., insulin diabetes); glomerulonephritis; graft versus host rejection; hemorrhagic shock; hyperalgesia, inflammatory bowel disease; inflammatory conditions of a joint including osteoarthritis, psoriatic arthritis and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS); multiple myeloma; multiple sclerosis; myelogenous (e.g., AML and CML) and other leukemias; myopathies (e.g., muscle protein metabolism, specifically in sepsis); osteoporosis; Parkinson's disease; chronic pain; pre-term labor; psoriasis; reperfusion injury; septic shock; side effects from radiation therapy, temporal mandibular joint disease, tumor metastasis; or an inflammatory condition resulting from strain, sprain, cartilage damage, trauma, orthopedic surgery, and infection.

### BRIEF DESCRIPTION OF THE FIGURES

The present invention is explained in greater detail in the description, figures and claims below.

FIG. 1A shows the sequence alignment of the novel splice variant CGEN-R1 (SEQ ID NO:2) with a previously known human interleukin-1 receptor antagonist (SEQ ID NO:8). The unique fragment of CGEN-R1 is marked in bold face.

FIG. 1B shows the sequence alignment of CGEN-R1 (SEQ ID NO:2) with a previously known human interleukin-1 receptor antagonist (SEQ ID NO:8) and with its splice variants (SEQ ID NOs:9, 10 and 1).

FIG. 2 Polynucleotide sequence of CGEN-R1 with signal peptide (SEQ ID NO:1) (A) and without signal peptide (SEQ ID NO:3) (B). Modified polynucleotide sequence of CGEN-R1 with signal peptide (SEQ ID NO:5) and its deduced amino acid sequence (SEQ ID NO:2) (C) and modified CGEN-R1 without signal peptide (SEQ ID NO:6) (D).

FIG. 3 Schematic presentation of primers used for examining the expression of CGEN-R1.

FIG. 4 RT-PCR results demonstrating tissue distribution of IL-1Ra and CGEN-R1 RNA expression.

FIG. 5 Expression of cloned IL-1Ra in bacteria. (A) Expression of wild type IL-1Ra. (B) Recognition of IL-1Ra and CGEN-R1 by commercially available anti-WT IL-1Ra antibody. (C) Recognition of CGEN-R1 by sera of immunized rabbits. (D-E) Expression of IL-1Ra and CGEN-R1. F. Purity and recognition by anti-CGEN-R1 sera of purified CGEN-R1.

FIG. 6A-C Inhibition of IL-1β-induced secretion of IL-8 from T24 cells by IL-1Ra and CGEN-R1.

FIG. 7 Expression of IL-1Ra and CGEN-R1 in HepG2 conditioned media.

### DETAILED DESCRIPTION OF THE PREFERRED EMBODIMENTS

The present invention provides (i) a novel Interleukin-1 Receptor antagonist which is referred to herein as CGEN-R1; (ii) polynucleotide sequences encoding CGEN-R1; (iii) oligonucleotides and oligonucleotide analogs derived from these polynucleotide sequences; (iv) antibodies recognizing said CGEN-R1; (v) peptides or peptide analogs derived from said CGEN-R1; and (vi) pharmaceutical compositions comprising any one of (i) to (iv); and (vii) methods of using the polypeptides, peptides or peptide analogs, the oligonucleotides and oligonucleotide analogs, and/or the polynucleotide sequences to regulate interleukin-1 activity.

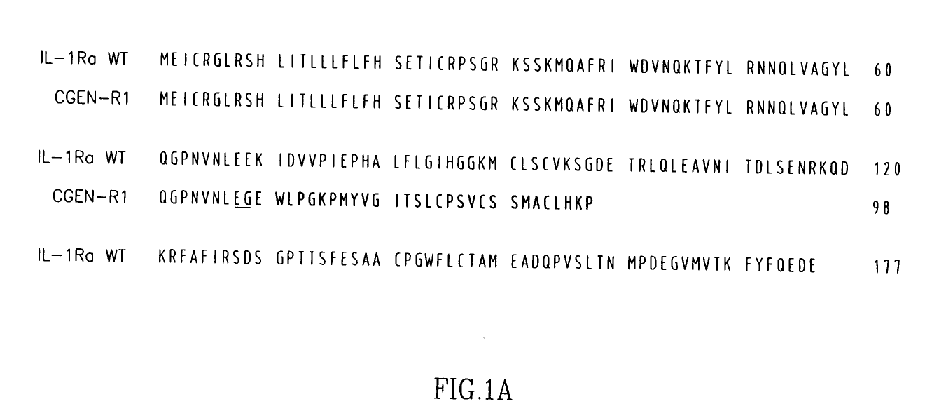

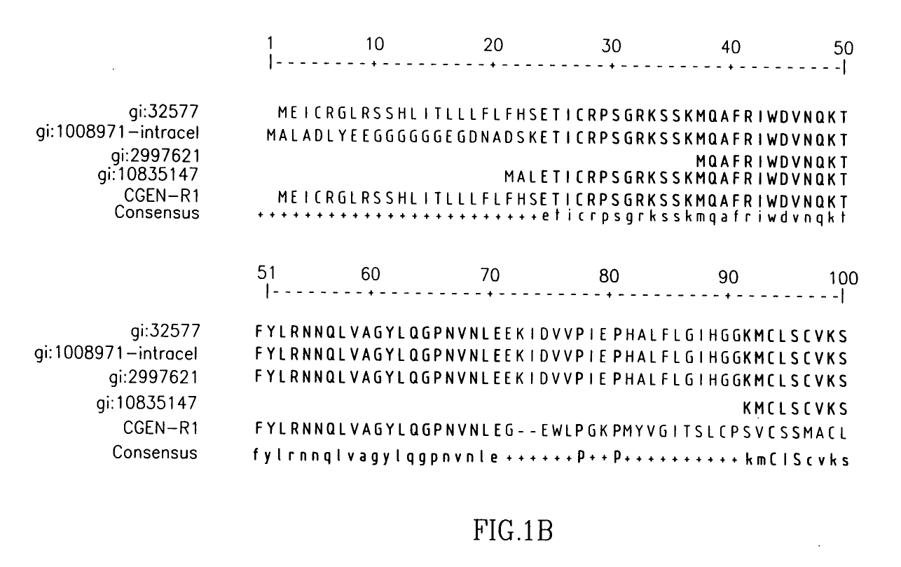

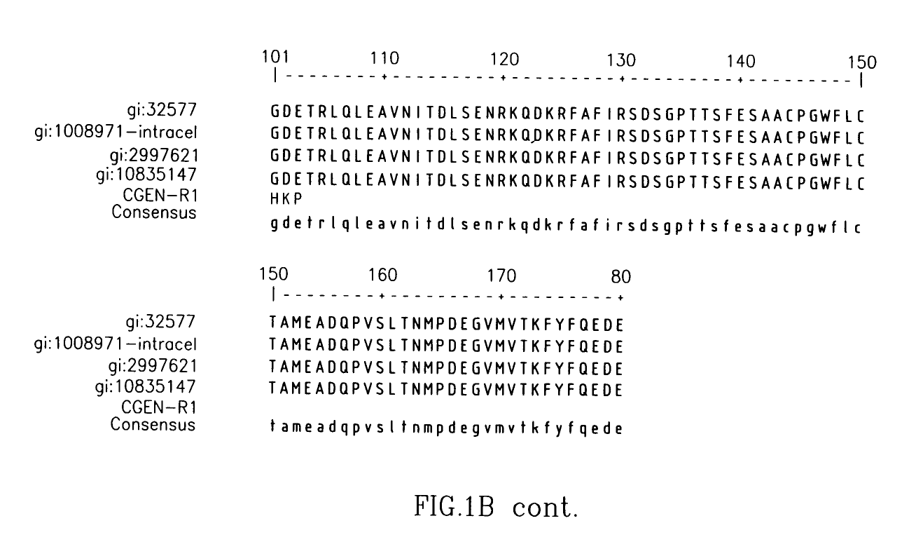

According to one preferred embodiment of the present invention, there is provided a CGEN-R1 protein, comprising a first portion having an amino acid sequence being at least about 90% homologous, and preferably at least about 95% homologous, to amino acids 1-68 of wild type IL-1Ra (SEQ ID NO:8; FIG. 1A), which are also amino acids 1-68 of SEQ ID NO:2; and a second portion having an amino acid sequence being at least about 80%, optionally at least about 85%, preferably at least about 90%, and more preferably at least about 95% homologous to an amino acid sequence GEWLPGKPMYVGITSLCPSVCSSMACLHKP, wherein the first and second portions are contiguous and in sequential order.

According to other preferred embodiments of the present invention, there is provided a tail portion of CGEN-R1 according to the present invention, which comprises a peptide according to an amino acid sequence GEWLPGKPMYVGITSLCPSVCSSMACLHKP, or a sequence at least about 80%, optionally at least about 85%, preferably at least about 90%, and more preferably at least about 95% homologous to this amino acid sequence. This peptide is present at amino acids 69-98 of CGEN-R1 with a signal peptide according to the present invention (SEQ ID NO:2).

According to another aspect, the novel splice variants of the present invention comprise a unique sequence in the region joining or bridging the novel tail (C-terminal) sequences comprising amino acids 69-98 of SEQ ID NO:2 (equivalent to residues 44-73 of SEQ ID NO:4) to the known subsequence comprising amino acids 1-68 (equivalent to 1-43 of SEQ ID NO:4) that is common to CGEN-R1 as set forth in SEQ ID NO:2 and the previously known IL-1Ra as set forth in SEQ ID NO:8. The unique joining or bridging region is a feature that characterizes the novel splice variants according to the present invention and distinguishes them from the previously known variants. This bridge portion can also comprise an epitope that is specific to the novel splice variants of the invention.

According to an additional preferred embodiment of the present invention, there is provided a bridge portion of SEQ ID NO:2, comprising a peptide sequence having a length “n”, wherein n is from about 4 to 50 amino acids, preferably from about 5 to 40 amino acids, more preferably 6-30 amino acids, the bridge portion comprising at least the dipeptide EG at positions 68-69 of SEQ ID NO:2 (marked with underlining on FIG. 1A), the bridge portion defined as follows (following the numbering set forth in SEQ ID NO:2): a sequence starting from any of amino acid numbers 68−x to 68; and ending at any of amino acid numbers 69+((n−2)−x), wherein x varies from 0 to n-2; wherein the total amino acid number does not exceed 98. For example, for peptides of 10 amino acids (such that n=10), the starting position could be as “early” in the sequence as amino acid number 60 if x=n−2=8 (ie 60=68−8), such that the peptide would end at amino acid number 69 (69+(8−8=0)). On the other hand, the peptide could start at amino acid number 68 if x=0 (ie 68=68−0), and could end at amino acid 77 (69+(8−0=8)).

According to another preferred embodiment, the bridge portion above optionally comprises a polypeptide being at least 80%, optionally at least about 85%, preferably at least about 90%, and more preferably at least about 95% homologous to at least one sequence described above.

Similarly, the bridge portion can optionally be relatively short, such as from about 4 to about 9 amino acids in length. For four amino acids, the first bridge portion would comprise the following peptides: EGEW; NLEG; LEGE. All peptides feature EG as a portion thereof. Peptides of from about five to about nine amino acids could optionally be similarly constructed.

It should be noted that CGEN-R1 according to the present invention clearly has a different amino acid sequence and a different nucleic acid sequence, as shown for example with regard to FIG. 1A (comparison to wild type IL-1Ra [SEQ ID NO:8] and 1B (comparison to wild type IL-1Ra [SEQ ID NO:8], and its splice variants gi:1008971 [SEQ ID NO:9], gi:2997621 [SEQ ID NO:10] and gi:10835147 [SEQ ID NO:11]. The comparison with regard to wild type IL-1Ra is described above, clearly emphasizing the differences between the splice variant of the present invention (which is a truncation of wild type IL-1Ra with a unique tail added) and the known protein.

With regard to the amino acid sequences shown in FIG. 1B, clearly the sequence of CGEN-R1 is more similar (in the portion before the unique tail) to wild type IL-1Ra than to the other sequences, and that furthermore, the unique tail sequence according to the present invention is not present in any other amino acid sequence shown. Indeed, the unique tail sequence is highly dissimilar to all other sequences of known variants, as well as being highly dissimilar to the sequence of wild type IL-1Ra.

While conceiving the present invention it was hypothesized that additional, previously unknown, IL-1Ra splice variants may exist. Splice variants, which may occur in over 50% of human genes, are usually overlooked in attempts to identify differentially expressed genes, as their unique sequence features including donor-acceptor concatenation, an alternative exon, an exon and a retained intron, complicate their identification. However, splice variants may have direct therapeutic utility and specific therapeutic profile distinct from their known counterparts. In addition, identification of splice variants may have an important impact on the understanding of disease development and may serve as valuable markers for various pathologies.

Novel splice variants were retrieved by screening a LEADS database with mRNA sequences of the known wild type IL-1Ra (gi:32576, SEQ ID NO:7) and its variants (gi:27894315, SEQ ID NO:14; gi: 1008970, SEQ ID NO:12; and gi:2997620, SEQ ID NO:13). The screening revealed three types of Expressed Sequence Tagged (EST) clones: An EST clone containing part of exon 1 and exon 2 joined to a unique sequence, derived from an intron 2 of the wild type IL-1Ra (SEQ ID NO:7). This EST was identified in a human cDNA library NIH_MGC_{—}120 (Pooled Pancreas and Spleen, Accession number B1836973); Four EST clones with high homology to the above unique intron-derived sequence were found to be transcribed, with the transcript having no homology to known IL-1Ra sequences, in the following human cDNA libraries: NCI_CGAP_GU1 (2 pooled high-grade transitional cell tumors) cDNA library (Accession number: AW630035); NT0022 (Nervous Tumor) cDNA library (Accession number: BF365244); Stratagene liver (#937224) (Liver) cDNA library (Accession number: T71181) and HT0125 (Head and Neck tumor) cDNA library (Accession number: AW178803); An EST clone originating from a human HT0366 (Head and Neck Tumor) cDNA library (Accession number BE706905) encoding a unique intron 2-derived sequence joined to exon 3 of the known IL-1Ra (SEQ ID NO:7).

All the above ESTs and cDNA sequences support the retention of intron 2 of wild type IL-1Ra (SEQ ID NO:7) in the coding sequence of the new splice variant of IL-1Ra.

While reducing the present invention to practice these clones have been characterized as encoding a previously unknown antagonist of the interleukin-1 receptor, which is referred to herein as CGEN-R1. This novel IL-1 receptor antagonist comprises as a C terminal portion a unique amino acid sequence sharing no homology to the known human IL-1Ra sequence of SEQ ID NO:8 (FIG. 1A), and apparently not sharing homology to any other known IL-1 receptor antagonists or any other known protein (see also FIG. 1B).

Before describing the present proteins, nucleotide sequences, the compositions and methods, it is to be understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to an “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

Definitions

Interleukin-1 receptor antagonist (IL-1Ra), as used herein, refers to the amino acid sequences of IL-1Ra obtained from any species, particularly mammalian, including bovine, ovine, porcine, murine, equine, and preferably human, from any source whether natural, synthetic, semi-synthetic, or recombinant.

An “allele” or “allelic sequence”, as used herein, is an alternative form of the gene encoding IL-1Ra. Alleles may result from at least one mutation in the nucleic acid sequence and may result in altered mRNAs or polypeptides whose structure or function may or may not be altered. Any given natural or recombinant gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

“Altered” nucleic acid sequences encoding IL-1Ra as used herein include those with deletions, insertions, or substitutions of different nucleotides resulting in a polynucleotide that encodes the same or a functionally equivalent IL-1Ra. Included within this definition are polymorphisms which may or may not be readily detectable using a particular oligonucleotide probe of the polynucleotide encoding IL-1Ra, and improper or unexpected hybridization to alleles, with a locus other than the normal chromosomal locus for the polynucleotide sequence encoding IL-1Ra. The encoded protein may also be “altered” and contain deletions, insertions, or substitutions of amino acid residues which produce a silent change and result in a functionally equivalent IL-1Ra. Deliberate amino acid substitutions may be made on the basis of similarity in polarity, charge, solubility, hydrophobicity, hydrophilicity, and/or the amphipathic nature of the residues as long as the biological or immunological activity of IL-1Ra is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

“Amino acid sequence”, as used herein, refers to an oligopeptide, peptide, polypeptide, or protein sequence, and fragment thereof, and to naturally occurring or synthetic molecules. Active fragments of IL-1Ra retain at least one biological activity or immunological activity or at least one antigenic epitope of IL-1Ra. Where “amino acid sequence” is recited herein to refer to an amino acid sequence of a protein molecule, amino acid sequence, and like terms, are not meant to limit the amino acid sequence to the complete, native amino acid sequence associated with the recited protein molecule.

“Amplification” as used herein refers to the production of additional copies of a nucleic acid sequence and is generally carried out using polymerase chain reaction (PCR) technologies well known in the art (Dieffenbach C W and G S Dveksler 1995 PCR Primer, a Laboratory Manual, Cold Spring Harbor Press, Plainview, N.Y.).

The term “antagonist”, as used herein, refers to a molecule which decreases the amount or the duration of the effect of the biological or immunological activity of IL-1. Antagonists may include proteins, nucleic acids, carbohydrates, antibodies or any other molecules which decrease the effect of IL-1.

“Antibody” refers to a polypeptide ligand substantially encoded by an immunoglobulin gene or immunoglobulin genes, or fragments thereof, which specifically binds and recognizes an epitope (e.g., an antigen). As used herein, this term refers to intact molecules as well as to fragments thereof, such as Fab, F(ab′)₂, Fv, scFv and the like which are capable of binding the epitopic determinant. Antibodies that bind IL-1Ra polypeptides can be prepared using intact polypeptides or fragments containing small peptides of interest as the immunizing antigen. The polypeptide or oligopeptide used to immunize an animal can be derived from the translation of RNA or synthesized chemically and can be conjugated to a carrier protein, if desired. Commonly used carriers that are chemically coupled to peptides are exemplified by bovine serum albumin, thyroglobulin and keyhole limpet hemocyanin. The coupled peptide is then used to immunize the animal (e.g., a mouse, a rat, or a rabbit).

The term “antigenic determinant”, as used herein, refers to that fragment of a molecule (i.e., an epitope) that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The term “antisense”, as used herein, refers to any composition containing nucleotide sequences which are complementary to a specific DNA or RNA sequence. The term “antisense strand” is used in reference to a nucleic acid strand that is complementary to the “sense” strand. Antisense molecules also include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand. Antisense oligonucleotides are also used for modulation of alternative splicing in vivo and for diagnostics in vivo and in vitro (Khelifi C., et al., Current Pharmaceutical Design, 2002, 8, 1451-1466; Sazani, P., and Kole. R. Progress in Molecular and Cellular Biology, 2003, 31: 217-239).

The term “biologically active”, as used herein, refers to a protein having structural, regulatory, or biochemical functions of a naturally occurring molecule. Likewise, “immunologically active” refers to the capability of the natural, recombinant, or synthetic IL-1Ra, or any oligopeptide thereof, to induce a specific immune response in appropriate animals or cells and to bind with specific antibodies.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of peptide nucleic acid (PNA) molecules.

As used herein, the phrase “complementary polynucleotide sequence” includes sequences which originally result from reverse transcription of messenger RNA using a reverse transcriptase or any other RNA dependent DNA polymerase. Such sequences can be subsequently amplified in vivo or in vitro using a DNA dependent DNA polymerase.

As used herein, the phrase “composite polynucleotide sequence” includes sequences which are at least partially complementary and at least partially genomic. A composite sequence can include some exonal sequences required to encode a polypeptide, as well as some intronic sequences interposed therebetween. The intronic sequences can be of any source, including of other genes, and typically will include conserved splicing signal sequences. Such intronic sequences may further include cis acting expression regulatory elements.

A “composition comprising a given polynucleotide sequence” as used herein refers broadly to any composition containing the given polynucleotide sequence. The composition may comprise a dry formulation or an aqueous solution. Compositions comprising polynucleotide sequences encoding CGEN-R1 (SEQ ID NO:2 or 4) or fragments thereof may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., SDS) and other components (e.g., Denhardt's solution, dry milk, salmon sperm DNA, etc.).

A “deletion”, as used herein, refers to a change in the amino acid or nucleotide sequence and results in the absence of one or more amino acid residues or nucleotides.

The term “derivative”, as used herein, refers to the chemical modification of a nucleic acid encoding or complementary to IL-1Ra or to the encoded IL-1Ra. Such modifications include, for example, replacement of hydrogen by an alkyl, acyl, or amino group. A nucleic acid derivative encodes a polypeptide which retains the biological or immunological function of the natural molecule. A derivative polypeptide is one which is modified by glycosylation, pegylation, or any similar process which retains the biological or immunological function of the polypeptide from which it was derived.

The phrase “differentially present” refers to differences in the quantity of a marker present in a sample taken from patients having the disease to be detected as compared to a comparable sample taken from healthy controls. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present.

As used herein the terms “diagnosing” or “diagnostic” refer to classifying a disease or a symptom, determining a severity of the disease, monitoring disease progression, forecasting an outcome of a disease and/or prospects of recovery. Diagnosis of a disease according to the present invention can be effected by determining a level of a polynucleotide or a polypeptide of the present invention in a biological sample obtained from the subject, wherein the level determined can be correlated with predisposition to, or presence or absence of the disease, or to its severity.

As used herein, the phrase “genomic polynucleotide sequence” includes sequences which originally derive from a chromosome and reflect a contiguous portion of a chromosome. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “homology”, as used herein, refers to a degree of sequence similarity in terms of shared amino acids or nucleotide sequences. There may be partial homology or complete homology (i.e., identity). The degree of homology may be determined using suitable software and parameters as are known to a person skilled in the art (e.g. Altschul S F Gish W Miller W Myers E W Lipman D J 1990 J Mol Biol 215(3): 403-10; Altschul S F Madden T L Schaffer A A Zhang J Zhang Z Miller W & Lipman D J 1997 Nucleic Acids Res. 25: 3389-3402).

The term “humanized antibody”, as used herein, refers to antibody molecules in which amino acids have been replaced in the non-antigen binding regions in order to more closely resemble a human antibody, while still retaining the original binding ability.

The term “hybridization”, as used herein, refers to any process by which a strand of nucleic acid binds with a complementary strand through base pairing.

“Immunoassay” is an assay that uses an antibody to specifically bind an antigen. The immunoassay is characterized by the use of specific binding properties of a particular antibody to isolate, target, and/or quantify the antigen. The phrase “specifically (or selectively) binds” to an antibody or “specifically (or selectively) immunoreactive with” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

An “insertion” or “addition”, as used herein, refers to a change in an amino acid or nucleotide sequence resulting in the addition of one or more amino acid residues or nucleotides, respectively, as compared to the disclosed molecules.

As used herein, the term “level” refers to expression levels of RNA and/or protein and/or anti-IL-1Ra splice variant antibody and/or antibody-antigen complexes or to DNA copy number of a marker of the present invention. The present invention preferably encompasses antibodies capable of selectively binding (with at least two fold higher binding) to at least one epitope of a IL-1Ra splice variant polypeptide according to the present invention as compared to any other polypeptide described herein, such as the previously described known variants and wild type (WT) IL-1Ra. The present invention also preferably encompasses any antibody-antigen complex formed with such antibodies and epitopes. Optionally and preferably, an epitope comprises a bridge of an amino acid sequence as described in the glossary.

Typically the level of the marker in a biological sample obtained from the subject is different (i.e., increased or decreased) from the level of the same variant in a similar sample obtained from a healthy individual.

The term “marker” in the context of the present invention refers to a nucleic acid fragment, a peptide, or a polypeptide, which is differentially present in a sample taken from patients having a particular disease or condition as compared to a comparable sample taken from subjects who do not have the particular disease or condition. A “test amount” of a marker refers to an amount of a marker present in a sample being tested. A test amount can be either in absolute amount (e.g., microgram/ml) or a relative amount (e.g., relative intensity of signals). A “control amount” of a marker can be any amount or a range of amounts to be compared against a test amount of a marker. A control amount can be either in absolute amount (e.g., microgram/ml) or a relative amount (e.g., relative intensity of signals). “Microarray” refers to an array of distinct polynucleotides or oligonucleotides synthesized on a substrate, such as paper, nylon or other type of membrane, filter, chip, glass slide, or any other suitable solid support.

The term “modulate”, as used herein, refers to a change in the activity of IL-1Ra. For example, modulation may cause an increase or a decrease in protein activity, binding characteristics, or any other biological, functional or immunological properties of IL-1Ra.

“Nucleic acid sequence” as used herein refers to an oligonucleotide, nucleotide, or polynucleotide, and fragments thereof, and to DNA or RNA of genomic or synthetic origin which may be single- or double-stranded, and represent the sense or antisense strand. “Fragments” are those nucleic acid sequences which are greater than 60 nucleotides in length, preferably at least 100 nucleotides in length.

The term “oligonucleotide” refers to a nucleic acid sequence of at least about 6 nucleotides to about 60 nucleotides, preferably about 15 to 30 nucleotides, and more preferably about 20 to 25 nucleotides, which can be used in PCR amplification or a hybridization assay, or a microarray. As used herein, oligonucleotide is substantially equivalent to the terms “amplimers”, “primers”, “oligomers”, and “probes”, as commonly defined in the art.

The term “peptide nucleic acid” (PNA) as used herein refers to nucleic acid “mimics”; the molecule's natural backbone is replaced by a pseudopeptide or peptide backbone and only the nucleotide base sequences are retained. The peptide backbone may end in lysine, which confers solubility to the composition. PNAs may be pegylated to extend their lifespan in the cell where they preferentially bind complementary single stranded DNA and RNA and stop transcript elongation.

The term “portion”, as used herein, with regard to a protein (as in “a portion of a given protein”) refers to fragments of that protein. The fragments may range in size from five amino acid residues to the entire amino acid sequence minus one amino acid. Thus, a protein “comprising at least a portion of the amino acid sequence of SEQ ID NO:2” encompasses the full-length CGEN-R1 and fragments thereof.

As used herein, a “primer” defines an oligonucleotide which is capable of annealing to a target sequence, thereby creating a double stranded region which can serve as an initiation point for DNA synthesis under suitable conditions.

The term “probe” refers to the IL-1Ra splice variant nucleic acid sequence, or a sequence complementary therewith, including bridge sequences and sequences complementary therewith, when used to detect presence of other similar sequences in a sample. The detection is carried out by identification of hybridization complexes between the probe and the assayed sequence. The probe may be attached to a solid support or to a detectable label.

The terms “sample” or “biological sample”, as used herein, are used in their broadest sense. A biological sample suspected of containing nucleic acid encoding IL-1Ra, or fragments thereof, or Il-1Ra itself may comprise a bodily fluid, extract from a cell, chromosome, organelle, or membrane isolated from a cell, a cell, genomic DNA, RNA, or cDNA in solution or bound to a solid support, a tissue, a tissue print, and the like. For example, a sample may include, but is not limited to, plasma, serum, spinal fluid, lymph fluid, the external sections of the skin, respiratory, intestinal, and genitourinary tracts, tears, saliva, milk, blood cells, tumors, neuronal tissue, organs, and also samples of in vivo cell culture constituents, amniotic fluid. A tissue sample would optionally and preferably include prostate tissue and/or other tissues of the male genitalia, or reproductive or urinary tracts. A fluid sample would optionally and preferably include blood (optionally including whole blood and/or blood fractions), semen or urine, for example. Numerous well known tissue or fluid collection methods can be utilized to collect the biological sample from the subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), as well as potentially less invasive methods such as lavage for example. Regardless of the procedure employed, once a biopsy is obtained the level of the variant can be determined and a diagnosis can thus be made. Determining the level of the same variant in normal tissues of the same origin is preferably effected along-side to detect an elevated expression and/or amplification. The terms “specific binding” or “specifically binding”, as used herein, refers to that interaction between a protein or peptide and an agonist, an antibody and an antagonist. The interaction is dependent upon the presence of a particular structure (i.e., the antigenic determinant or epitope) of the protein recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

As used herein “splice variants” refers to nucleic acid sequences and proteins encoded therefrom which are products of alternative splicing. Alternative splicing refers to intron inclusion, exon exclusion, or any addition or deletion of terminal sequences, which results in sequence dissimilarities between the splice variant sequence and the wild-type sequence. Although most alternatively spliced variants result from alternative exon usage, some result from the retention of introns not spliced-out in the intermediate stage of RNA transcript processing.

The terms “stringent conditions” or “stringency”, as used herein, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. to about 25° C. below the melting temperature of the probe). One or more factors may be varied to generate conditions of either low or high stringency.

The term “substantially purified”, as used herein, refers to nucleic or amino acid sequences that are removed from their natural environment, isolated or separated, and are at least 60% free, preferably 75% free, and most preferably 90% free from other components with which they are naturally associated.

A “substitution”, as used herein, refers to the replacement of one or more amino acids or nucleotides by different amino acids or nucleotides, respectively.

“Transformation”, as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

A “variant” of IL-1Ra, as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Analogous minor variations may also include amino acid deletions or insertions, or both. Guidance in determining which amino acid residues may be substituted, inserted, or deleted without abolishing biological or immunological activity may be found using computer programs well known in the art, for example, DNASTAR software.

Novel Splice Variants of IL-1Ra

Thus, according to one aspect of the present invention there is provided an isolated polynucleotide comprising a genomic, complementary or composite polynucleotide sequence encoding a novel splice variant IL-1 receptor antagonist polypeptide, i.e., capable of binding to a mammalian IL-1 receptor while being substantially devoid of IL-1 activity. In other words, the IL-1Ra binds to the IL-1 receptor but does not elicit activation of the signaling pathways evoked by IL-1.

Thus, according to one aspect of the present invention there is provided an isolated polynucleotide comprising a genomic, complementary or composite polynucleotide sequence encoding a polypeptide capable of binding to a mammalian IL-1 receptor which is at least 80%, preferably at least 85%, more preferably at least 90% or more, most preferably at least 95%, or more homologous (similar+identical amino acids) to SEQ ID NOs: 2 or 4. Preferably, the encoded polypeptide is substantially devoid of IL-1 activity.

According to one preferred embodiment the polynucleotide according to this aspect of the present invention encodes a polypeptide comprising contiguous amino acids having at least 80%, preferably at least 90%, more preferably 95% or more homology (similar+identical amino acids) to positions 44 to 73 of SEQ ID NO:4.

According to one embodiments, the polynucleotide according to another aspect of the present invention encodes a polypeptide as set forth in SEQ ID NOs: 2 or 4 or a portion thereof, which retains at least one biological, immunological or other functional characteristic or activity of IL-1Ra.

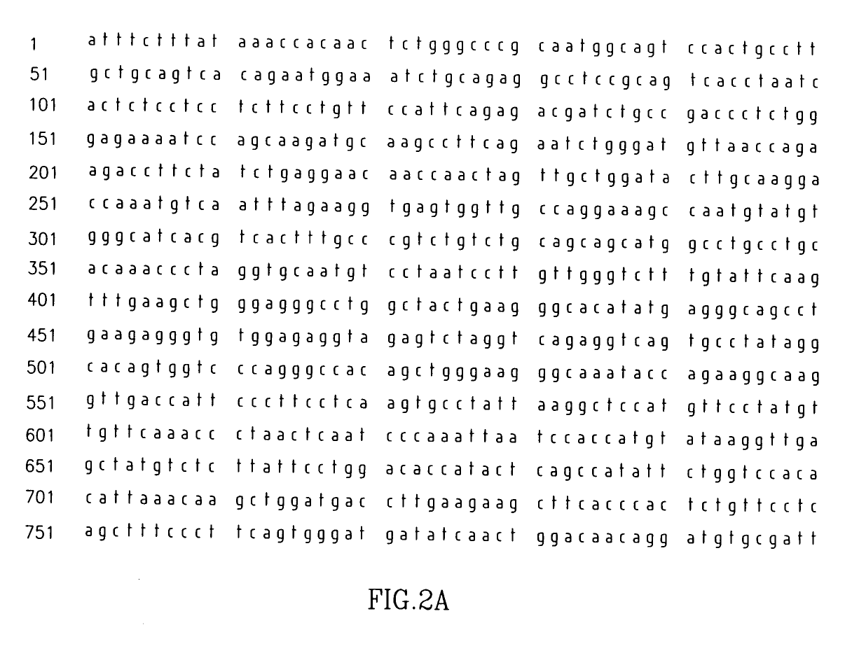

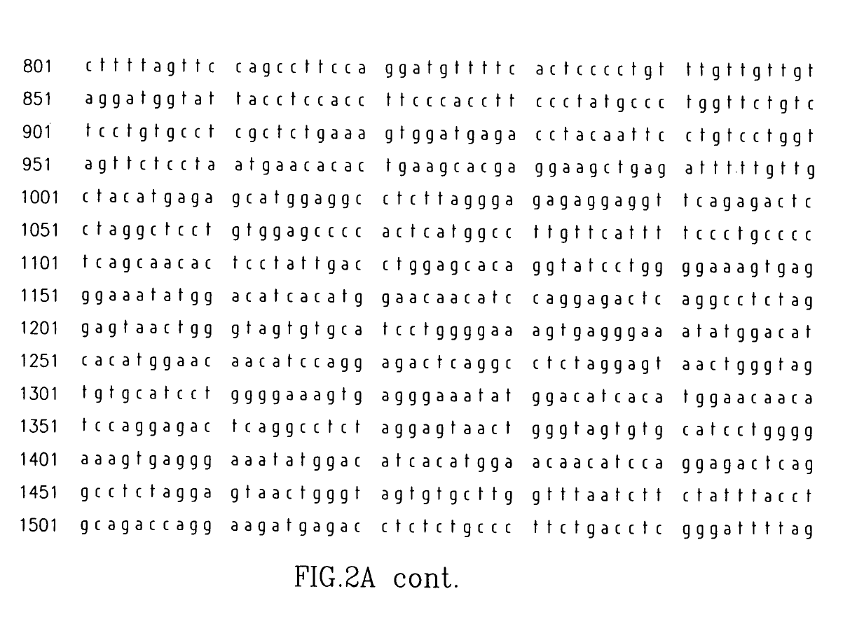

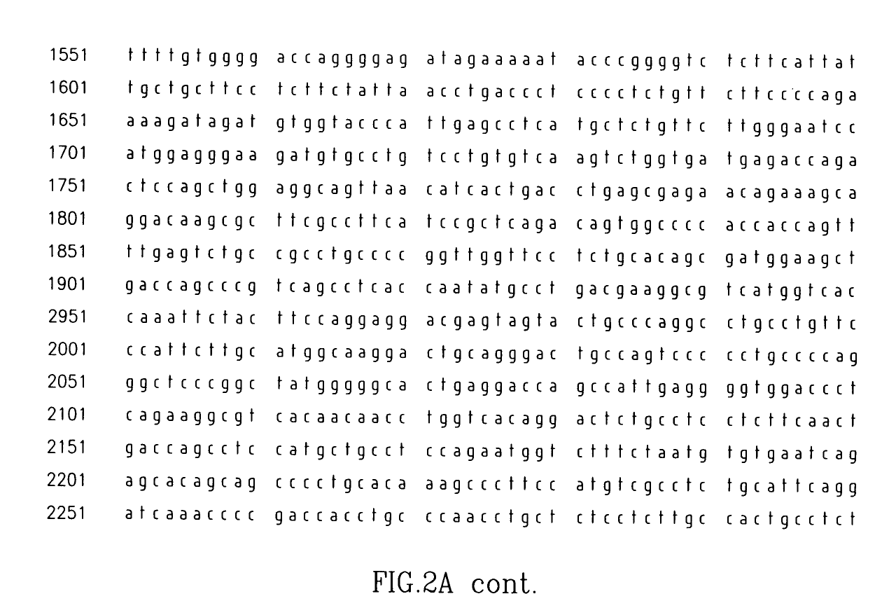

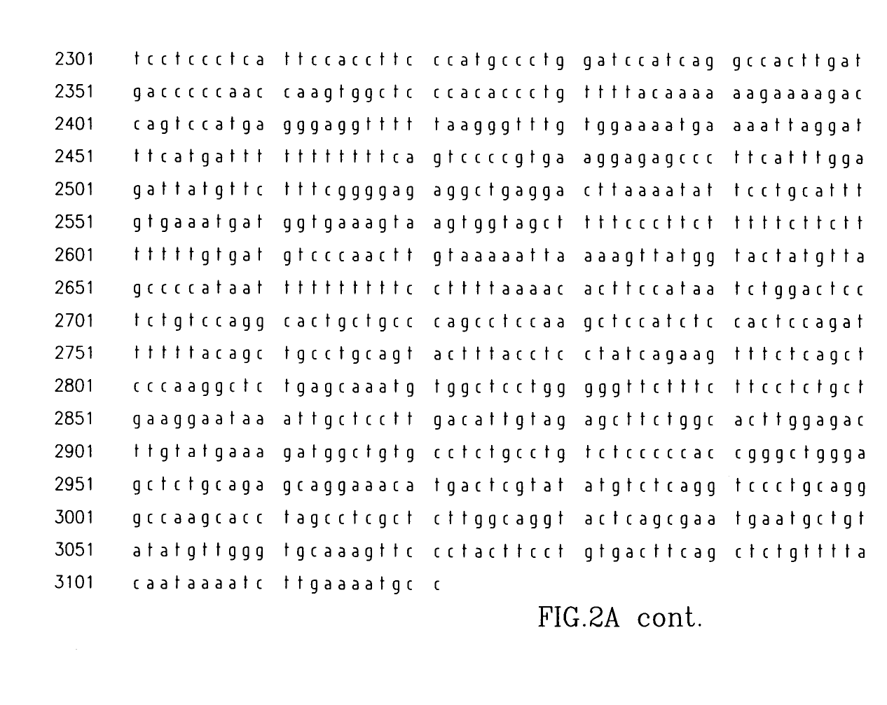

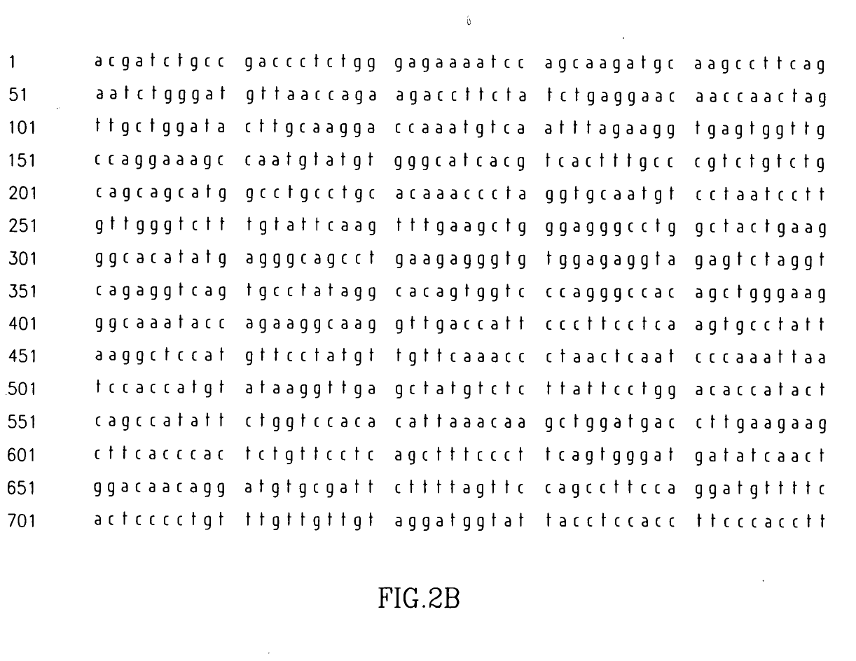

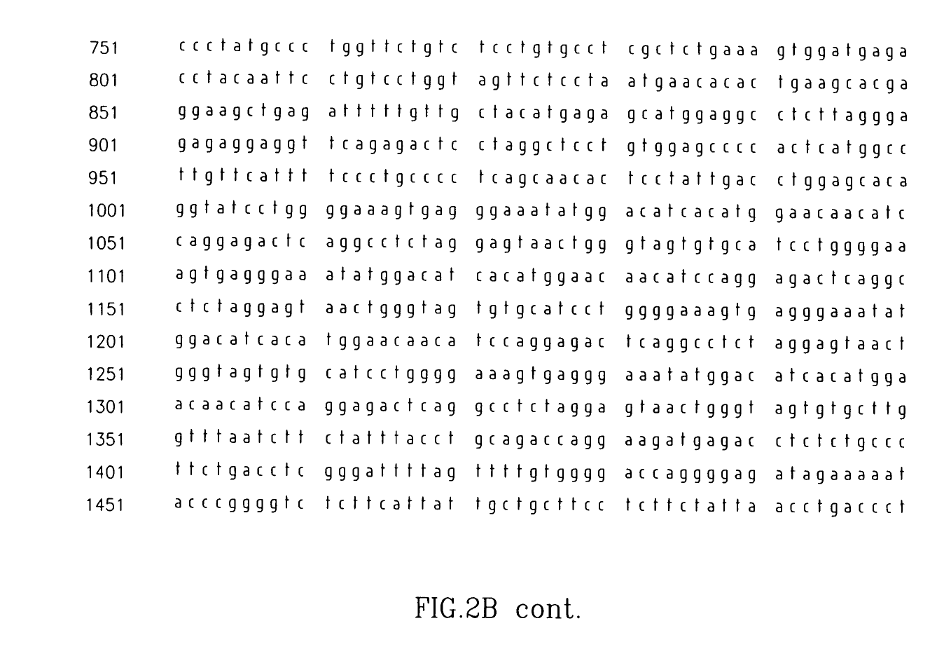

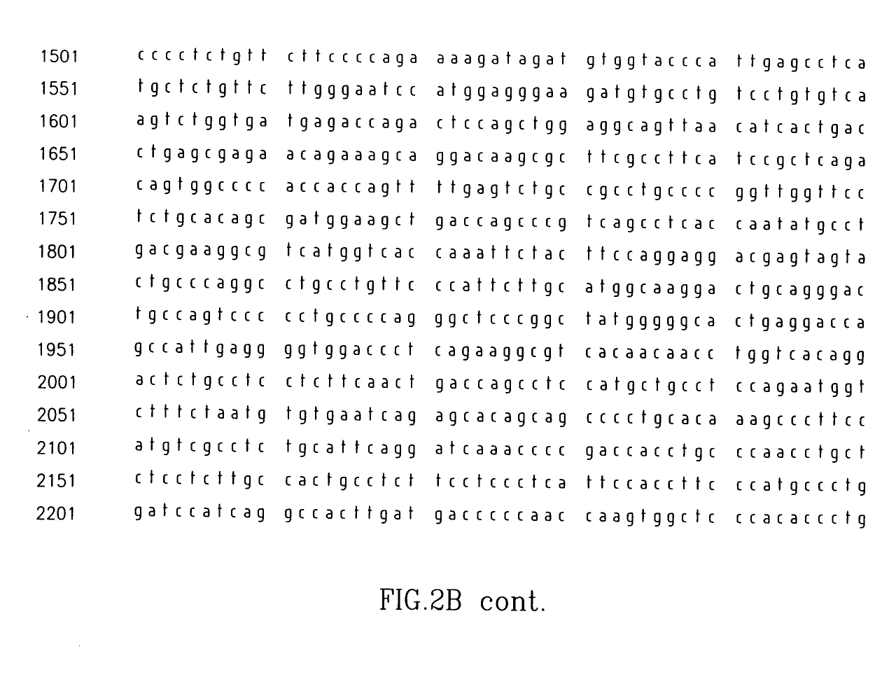

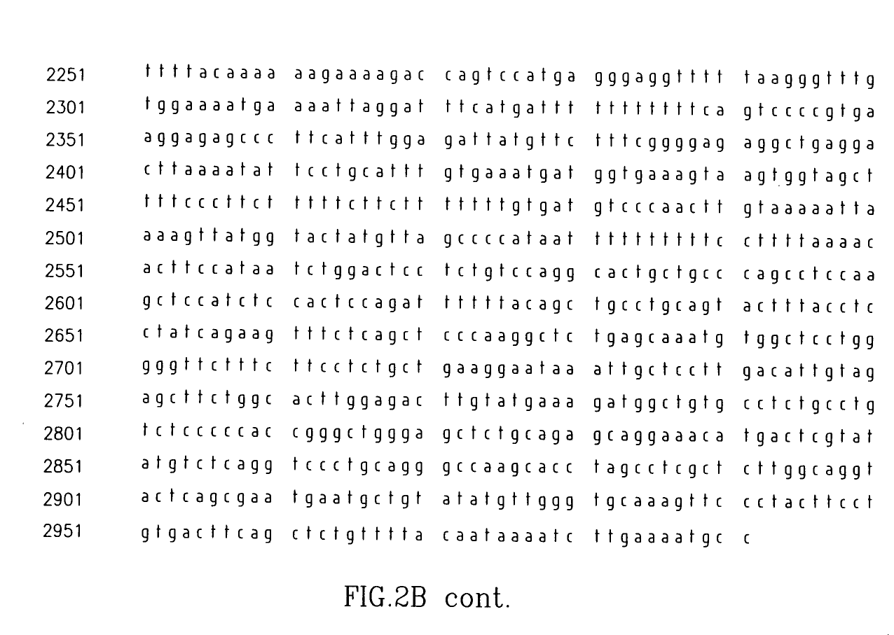

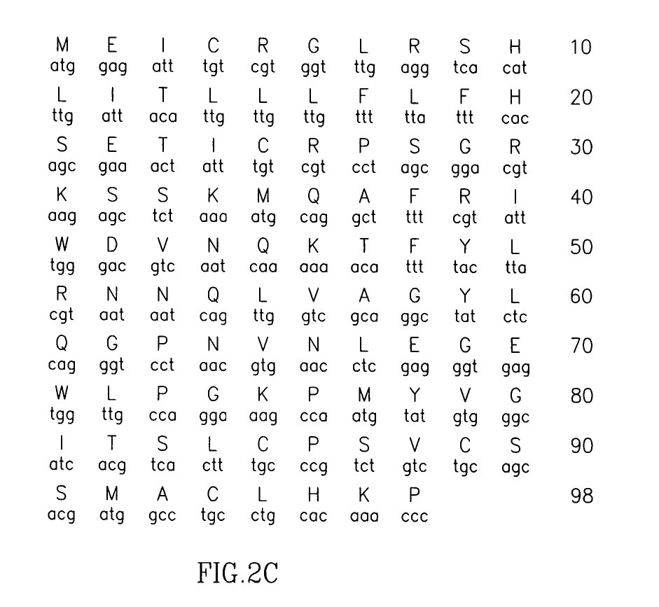

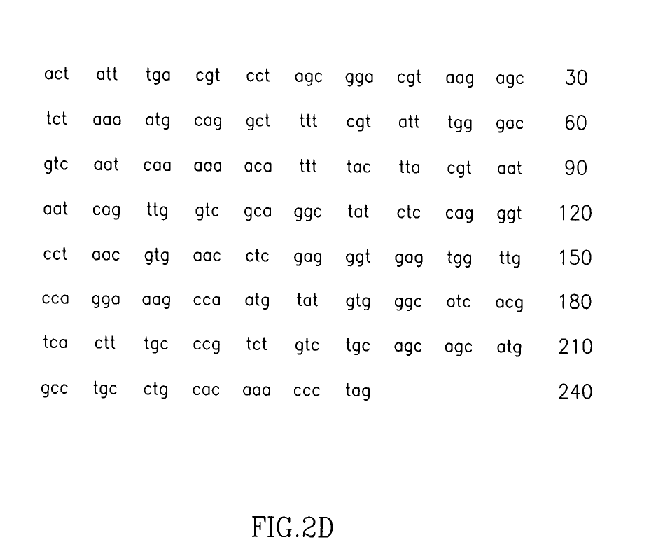

FIG. 2A shows the polynucleotide sequence of CGEN-R1 with signal peptide (SEQ ID NO:1). FIG. 2B shows this polynucleotide sequence without signal peptide (SEQ ID NO:3). FIG. 2C shows the modified polynucleotide sequence of CGEN-R1 with signal peptide (SEQ ID NO:5) and its deduced amino acid sequence (SEQ ID NO:2), while FIG. 2D shows the same for modified CGEN-R1 without signal peptide (SEQ ID NO:6).

Methods for DNA sequencing are well known and generally available in the art, and may be used to practice any of the embodiments of the invention. The methods may employ such enzymes as the Klenow fragment of DNA polymerase I, Sequenase™ (U.S. Biochemical Corp, Cleveland, Ohio), Taq polymerase (Perkin Elmer), thermostable T7 polymerase (Amersham, Chicago, Ill.), or combinations of polymerases and proofreading exonucleases such as those found in the ELONGASE Amplification System marketed by Gibco/BRL (Gaithersburg, Md.). Preferably, the process is automated with machines such as the Hamilton Micro Lab 2200 (Hamilton, Reno, Nev.), Peltier Thermal Cycler (PTC200; M J Research, Watertown, Mass.) and the ABI Catalyst and 373 and 377 DNA Sequencers (Perkin Elmer). It will be appreciated by those skilled in the art that as a result of the degeneracy of the genetic code, a multitude of nucleotide sequences encoding the novel CGEN-R1, some bearing minimal homology to the nucleotide sequences of any known and naturally occurring genes, may be produced. Thus, the invention contemplates each and every possible variation of nucleotide sequence that could be made by selecting combinations based on possible codon choices. These combinations are made in accordance with the standard triplet genetic code as applied to the nucleotide sequence of the novel CGEN-R1, and all such variations are to be considered as being specifically disclosed.

According to one embodiment, the isolated polynucleotides of the present invention include a polynucleotide comprising a nucleotide sequence selected from the group consisting of SEQ ID NOs:1, 3, 5 or 6.

Although nucleotide sequences which encode CGEN-R1 and its variants are preferably capable of hybridizing to the nucleotide sequence of the CGEN-R1 under appropriately selected conditions of stringency, it may be advantageous to produce nucleotide sequences encoding CGEN-R1 or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding CGEN-R1 and its derivatives without altering the encoded amino acid sequences include the production of RNA transcripts having more desirable properties, such as a greater half-life, than transcripts produced from the naturally occurring sequence.

The invention also encompasses production of DNA sequences, or fragments thereof, which encode CGEN-R1 and its derivatives, entirely by synthetic chemistry. After production, the synthetic sequence may be inserted into any of the many available expression vectors and cell systems using reagents that are well known in the art. Moreover, synthetic chemistry may be used to introduce mutations into a sequence encoding CGEN-R1 or any fragment thereof.

The present invention also includes polynucleotide sequences that are capable of hybridizing to the nucleotide sequences according to the present invention.

According to one embodiment, the polynucleotide is preferably hybridizable with a polynucleotide sequence selected from the group consisting of SEQ ID NOs: 1, 3, 5 or 6.

Hybridization for long nucleic acids (e.g., above 200 bp in length) is effected according to preferred embodiments of the present invention by stringent or moderate hybridization. For example, stringent hybridization may be effected by hybridization at 65° C. with a hybridization solution containing 1% SDS, with a final wash solution of 0.2×SSC and 0.1% SDS at 65° C. Moderate hybridization may be effected by a hybridization solution containing 1% SDS at hybridization temperature of 65° C., with a final wash with a solution of 1×SSC and 0.1% SDS at 50° C.

According to preferred embodiments the polynucleotide according to this aspect of the present invention is as set forth in SEQ ID NOs: 1, 3, 5, or 6 or a portion thereof, said portion preferably encodes a polypeptide comprising contiguous amino acids having at least 80%, preferably at least 90%, more preferably 95% or more homology (similar+identical amino acids) to positions 44 to 73 of SEQ ID NO:4.

According to still another embodiment of the present invention there is provided an oligonucleotide of at least 17, at least 18, at least 19, at least 20, at least 22, at least 25, at least 30 or at least 40, bases specifically hybridizable with the isolated nucleic acid described herein.

Hybridization of shorter nucleic acids (below 200 bp in length, e.g., 17-40 bp in length) is effected by stringent, moderate or mild hybridization. For example, stringent hybridization may be effected by a hybridization solution of 6×SSC and 1% SDS at hybridization temperature of 1-1.5° C. below the Tₘ, and final wash with solution of 3 M TMACI, 0.01 M sodium phosphate (pH 6.8), 0.5% SDS at 1-1.5° C. below the Tₘ. Moderate hybridization may be effected by a hybridization solution of 6×SSC, 0.1% SDS at hybridization temperature of 2-2.5° C. below the Tₘ, with final wash solution of 6×SSC at 22° C.; mild hybridization may be effected by a hybridization solution of 6×SSC and 1% SDS at 37° C., and final wash with solution of 6×SSC at 22° C.

According to an additional aspect of the present invention there is provided a pair of oligonucleotides each independently of at least 17-40 bases specifically hybridizable with the isolated nucleic acid described herein in an opposite orientation so as to direct exponential amplification of a portion thereof, in a nucleic acid amplification reaction, such as a polymerase chain reaction. The polymerase chain reaction and other nucleic acid amplification reactions are well known in the art and require no further description herein. The pair of oligonucleotides according to this aspect of the present invention are preferably selected to have comparable melting temperatures (Tₘ), e.g., melting temperatures which differ by less than that 7° C., preferably less than 5° C., more preferably less than 4° C., most preferably less than 3° C., ideally between 3° C. and 0° C. Consequently, according to yet an additional aspect of the present invention there is provided a nucleic acid amplification product obtained using the pair of primers described herein. Such a nucleic acid amplification product can be isolated by gel electrophoresis or by any other size-based separation technique. Alternatively, such a nucleic acid amplification product can be isolated by affinity separation, either stranded affinity or sequence affinity. In addition, once isolated, such a product can be further genetically manipulated by restriction, ligation and the like, to serve any one of a plurality of applications associated with regulation of IL-1 activity as further detailed herein.

The nucleic acid sequences encoding CGEN-R1 may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar G Turner R T Bolander M E 1993 PCR Methods Appl. 2: 318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region. The primers may be designed using commercially available software such as OLIGO 4.06 Primer Analysis software (National Biosciences Inc., Plymouth, Minn.), or another appropriate program, to be 22-30 nucleotides in length, to have a GC content of 50% or more, and to anneal to the target sequence at temperatures about 68° C. to 72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom M Parik J Malmgren H Stewart J Pettersson U Landegren U 1991 PCR Methods Appl. 1: 111-119). In this method, multiple restriction enzyme digestions and ligations may also be used to place an engineered double-stranded sequence into an unknown fragment of the DNA molecule before performing PCR.

When screening for full-length cDNAs, it is preferable to use libraries that have been size-selected to include larger cDNAs. Also, random-primed libraries are preferable, in that they will contain more sequences which contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory region.

According to one embodiment, cDNA libraries are generated from specific tissue types, for EST sequencing. Basically, after a cDNA library from a tissue of interest is created, clones are randomly picked from these libraries and then single sequencing reactions from a large number of clones are performed. Each sequencing reaction generates about 300 base pairs of sequence that represents a unique sequence tag for a particular transcript.

Capillary electrophoresis systems which are commercially available may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled devise camera. Output/light intensity may be converted to electrical signal using appropriate software (e.g. Genotyper™ and Sequence Navigator™, Perkin Elmer) and the entire process from loading of samples to computer analysis and electronic data display may be computer controlled. Capillary electrophoresis is especially preferable for the sequencing of small pieces of DNA which might be present in limited amounts in a particular sample. In summary, this aspect of the present invention encompasses (i) polynucleotide sequences selected from the group consisting of SEQ ID NOs: 1, 3, 5, or 6; (ii) fragments thereof; (iii) sequences hybridizable therewith; (iv) sequences homologous thereto; (v) sequences encoding similar polypeptides with different codon usage; (vi) altered sequences characterized by mutations, such as deletion, insertion or substitution of one or more nucleotides, either naturally occurring or man induced, either randomly or in a targeted fashion.

According to another aspect, the present invention provides a polypeptide which is a novel variant of IL-1 receptor antagonist.

According to one embodiment, the present invention provides a polypeptide comprising an amino acid sequence selected from the group consisting of SEQ ID NO:2, SEQ ID NO:4 and fragment, variant and analogs thereof, with the proviso that the polypeptide binds to a mammalian IL-1 receptor. Preferably, the polypeptide is substantially devoid of IL-1 activity.

According to one embodiment, the present invention provides a polypeptide having an amino acid sequence which is at least 80%, preferably at least 85%, more preferably at least 90%, most preferably at least 95% homologous (similar+identical amino acids) to SEQ ID NO:2 or 4.

According to yet another embodiment, the polypeptide comprises contiguous amino acids having at least 80%, preferably at least 90%, more preferably 95% or more homology (similar+identical amino acids) to positions 44 to 73 of SEQ ID NO:4.

Producing the Novel Variants

Constructs Comprising the Novel Variants

According to another aspect of the present invention there is provided a nucleic acid construct comprising the isolated nucleic acid described herein.

According to a preferred embodiment the nucleic acid construct according to this aspect of the present invention further comprises a promoter for regulating the expression of the isolated nucleic acid in a sense or antisense orientation. Such promoters are known to be cis-acting sequence elements required for transcription as they serve to bind DNA dependent RNA polymerase which transcribes sequences present downstream thereof. Such down stream sequences can be in either one of two possible orientations to result in the transcription of sense RNA which is translatable by the ribosome machinery or antisense RNA which typically does not contain translatable sequences, yet can duplex or triplex with endogenous sequences, either mRNA or chromosomal DNA and hamper gene expression, all as is further detailed hereinunder.

While the isolated nucleic acid described herein is an essential element of the invention, it is modular and can be used in different contexts. The promoter of choice that is used in conjunction with this invention is of secondary importance, and will comprise any suitable promoter sequence. It will be appreciated by one skilled in the art, however, that it is necessary to make sure that the transcription start site(s) will be located upstream of an open reading frame. In a preferred embodiment of the present invention, the promoter that is selected comprises an element that is active in the particular host cells of interest. These elements may be selected from transcriptional regulators that activate the transcription of genes essential for the survival of these cells in conditions of stress or starvation, including the heat shock proteins.

Vectors and Host Cells

In order to express a biologically active CGEN-R1, the nucleotide sequences encoding CGEN-R1 or functional equivalents according to the present invention may be inserted into appropriate expression vector, i.e., a vector which contains the necessary elements for the transcription and translation of the inserted coding sequence.

Vectors can be introduced into cells or tissues by any one of a variety of known methods within the art, including in vitro recombinant DNA techniques, synthetic techniques, and in vivo genetic recombination. Such methods are generally described, for example, in Sambrook et al., Molecular Cloning: A Laboratory Manual, Cold Springs Harbor Laboratory, New York 1989, 1992; in Ausubel et al., Current Protocols in Molecular Biology, John Wiley and Sons, Baltimore, Md. 1989; Chang et al., Somatic Gene Therapy, CRC Press, Ann Arbor, Mich. 1995; Vega et al., Gene Targeting, CRC Press, Ann Arbor Mich. 1995; Vectors: A Survey of Molecular Cloning Vectors and Their Uses, Butterworths, Boston Mass. 1988; and include, for example, stable or transient transfection, lipofection, electroporation and infection with recombinant viral vectors. In addition, U.S. Pat. Nos. 5,464,764 and 5,487,992 disclose positive-negative selection methods.

A variety of expression vector/host systems may be utilized to contain and express sequences encoding CGEN-R1. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems. The invention is not limited by the host cell employed. The expression of the construct according to the present invention within the host cell may be transient or it may be stably integrated in the genome thereof.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The “control elements” or “regulatory sequences” are those non-translated regions of the vector-enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the Bluescript^{RTM} phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding CGEN-R1, vectors based on SV40 or EBV may be used with an appropriate selectable marker.

In bacterial systems, a number of expression vectors may be selected depending upon the use intended for CGEN-R1. For example, when large quantities of CGEN-R1 are needed for the induction of antibodies, vectors which direct high level expression of fusion proteins that are readily purified may be used. Such vectors include, but are not limited to, the multifunctional E. coli cloning and expression vectors such as Bluescript^{RTM} (Stratagene), in which the sequence encoding CGEN-R1 may be ligated into the vector in frame with sequences for the amino-terminal Met and the subsequent 7 residues of β-galactosidase so that a hybrid protein is produced; pIN vectors (Van Heeke G and S M Schuster 1989 J Biol Chem 264: 5503-5509); and the like. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. (Reviewed by, e.g., Ausubel et al. (supra)).

In cases where plant expression vectors are used, the expression of sequences encoding CGEN-R1 may be driven by any of a number of promoters. For example, viral promoters such as the ³⁵S and 19S promoters of CaMV may be used alone or in combination with the omega leader sequence from TMV. Alternatively, plant promoters such as the small subunit of RUBISCO or heat shock promoters may be used. These constructs can be introduced into plant cells by direct DNA transformation or pathogen-mediated transfection. Such techniques are described in a number of generally available reviews (for example, Hobbs, S. or Murry, L. E. in McGraw Hill Yearbook of Science and Technology (1992) McGraw Hill, New York, N.Y.; pp. 191-196).

An insect system may also be used to express CGEN-R1. For example, in one such system, Autographa californica nuclear polyhedrosis virus (AcNPV) is used as a vector to express foreign genes in Spodoptera frugiperda cells or in Trichoplusia larvae. The sequences encoding CGEN-R1 may be cloned into a non-essential region of the virus, such as the polyhedrin gene, and placed under control of the polyhedrin promoter. Successful insertion of CGEN-R1 will render the polyhedrin gene inactive and produce recombinant virus lacking coat protein. The recombinant viruses may then be used to infect, for example, S. frugiperda cells or Trichoplusia larvae in which CGEN-R1 may be expressed (Engelhard E K Kam-Morgan L N, Washburn J O Volkman L E 1994 Proc. Nat. Acad. Sci. 91: 3224-3227).

In mammalian host cells, a number of viral-based expression systems may be utilized. In cases where an adenovirus is used as an expression vector, sequences encoding CGEN-R1 may be ligated into an adenovirus transcription/translation complex consisting of the late promoter and tripartite leader sequence. Insertion in a non-essential E1 or E3 region of the viral genome may be used to obtain a viable virus which is capable of expressing CGEN-R1 in infected host cells (Logan J and Shenk T 1984 Proc. Natl. Acad. Sci. 81: 3655-3659). In addition, transcription enhancers, such as the Rous sarcoma virus (RSV) enhancer, may be used to increase expression in mammalian host cells.

Human artificial chromosomes (HACs) may also be employed to deliver larger fragments of DNA than can be contained and expressed in a plasmid. HACs of 6 to 10M are constructed and delivered via conventional delivery methods (liposomes, polycationic amino polymers, or vesicles) for therapeutic purposes.

Specific initiation signals may also be used to achieve more efficient translation of sequences encoding CGEN-R1. Such signals include the ATG initiation codon and adjacent sequences. In cases where sequences encoding CGEN-R1, its initiation codon, and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf K D Materna T Treuter E Nover L 1994 Results Probl. Cell Differ. 20: 125-162).

Polypeptide Purification

Host cells transformed with nucleotide sequences encoding CGEN-R1 may be cultured under conditions suitable for the expression and recovery of the protein from cell culture. The protein produced by a transformed cell may be secreted or contained intracellularly depending on the sequence and/or the vector used. The polynucleotide encoding for CGEN-R1 may include a signal peptide (amino acid residues 1-25 of SEQ ID NO:2) which direct secretion of CGEN-R1 through a prokaryotic or eukaryotic cell membrane. Other constructions may be used to join sequences encoding CGEN-R1 to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.). The inclusion of cleavable linker sequences, such as those specific for Factor XA or enterokinase (Invitrogen, San Diego, Calif.), between the purification domain and the CGEN-R1 encoding sequence may be used to facilitate purification. One such expression vector provides for expression of a fusion protein containing CGEN-R1 and a nucleic acid encoding 6 histidine residues preceding a thioredoxin or an enterokinase cleavage site. The histidine residues facilitate purification on immobilized metal ion affinity chromatography. (IMIAC) (See, e.g., Porath J 1992 Prot. Exp. Purif. 3: 263-281.) The enterokinase cleavage site provides a means for purifying CGEN-R1 from the fusion protein. (See, e.g., Kroll D J Abdel-Malek Abdel-Hafiz H Marcell T Simpson S Chen C Y Gutierrez-Hartmann A Lustbader J W Hoeffler J P 1993 DNA Cell Biol. 12: 441-453.)

Fragments of CGEN-R1 may be produced not only by recombinant production, but also by direct peptide synthesis using solid-phase techniques. Protein synthesis may be performed by manual techniques or by automation. Automated synthesis may be achieved, for example, using the Applied Biosystems 431A peptide synthesizer (Perkin Elmer). Various fragments of CGEN-R1 may be synthesized separately and then combined to produce the full-length molecule.

Proteins

According to yet a further aspect of the present invention there is provided a recombinant or synthetic (i.e., prepared using solid phase peptide synthesis) protein comprising a polypeptide capable of binding to IL-1 receptor and which is at least 80%, preferably at least 85%, more preferably at least 90% or more, most preferably at least 95% or more homologous (similar+identical amino acids) to SEQ ID NOs: 2 or 4.

According to one preferred embodiment the protein comprises contiguous amino acids having at least 80%, preferably at least 90%, more preferably 95% or more homology (similar+identical amino acids) to positions 44 to 73 of SEQ ID NO:4.

Additionally or alternatively, the polypeptide according to this aspect of the present invention is preferably encoded by a polynucleotide hybridizable with SEQ ID NOs: 1, 3, 5, or 6 or a portion thereof under any of stringent or moderate hybridization conditions. Still additionally or alternatively, the polypeptide according to this aspect of the present invention is preferably encoded by a polynucleotide sequence selected from the group consisting of SEQ ID NOs:1, 3, 5, or 6 or portions thereof.

Thus, this aspect of the present invention encompasses (i) polypeptides as set forth in SEQ ID NOs: 2 or 4; (ii) fragments thereof comprising contiguous amino acids having at least 80%, preferably at least 90%, more preferably 95% or more homology (similar+identical amino acids) to positions 44 to 73 of SEQ ID NO:4; (iii) polypeptides homologous thereto; and (iv) altered polypeptide characterized by mutations, such as deletion, insertion or substitution of one or more amino acids, either naturally occurring or man induced, either in random or in a targeted fashion, either natural, non-natural or modified at or after synthesis.

According to still a further aspect the present invention provides a pharmaceutical composition comprising as an active ingredient the recombinant protein according to the present invention as described herein, and a pharmaceutically acceptable diluent or carrier which is further described above.

Peptides

Peptides according to the present invention preferably comprise peptides according to the tail and/or bridge portions of CGEN-R1, as described above.

As used the phrase “derived from a polypeptide” refers to peptides derived from the specified protein or proteins and further to homologous peptides derived from equivalent regions of proteins homologous to the specified proteins of the same or other species. The term further relates to permissible amino acid alterations and peptidomimetics designed based on the amino acid sequence of the specified proteins or their homologous proteins.

As used herein in the specification and in the claims section below the term “amino acid” is understood to include the 20 naturally occurring amino acids; those amino acids often modified post-translationally in vivo, including for example hydroxyproline, phosphoserine and phosphothreonine; and other unusual amino acids including, but not limited to, 2-aminoadipic acid: hydroxylysine isodesmosine, nor-valine, nor-leucine and ornithine. Furthermore, the term “amino acid” includes both D- and L-amino acids. Further elaboration of the possible amino acids usable according to the present invention and examples of non-natural amino acids are given hereinunder.

Hydrophilic aliphatic natural amino acids can be substituted by synthetic amino acids, preferably Nleu, Nval and/or α-aminobutyric acid or by aliphatic amino acids of the general formula HN(CH₂)ₙ COOH, wherein n=3-5, as well as by branched derivatives thereof, wherein an alkyl group, for example, methyl, ethyl or propyl, is located at any one or more of the n carbons.

Each one, or more, of the amino acids can include a D-isomer thereof. Positively charged aliphatic carboxylic acids, such as, but not limited to, H₂N(CH₂)ₙ COOH, wherein n=2-4 and H₂N—C(NH)—NH(CH₂)ₙCOOH, wherein n=2-3, as well as by hydroxy Lysine, N-methyl Lysine or ornithine (Orn) can also be employed. Additionally, enlarged aromatic residues, such as, but not limited to, H₂N—(C₆H₆)—CH₂—COOH, p-aminophenyl alanine, H₂N—F(NH)—NH—(C₆H₆)—CH₂—COOH, p-guanidinophenyl alanine or pyridinoalanine (Pal) can also be employed. Side chains of amino acid derivatives (if these are Ser, Tyr, Lys, Cys or Orn) can be protected-attached to alkyl, aryl, alkyloyl or aryloyl moieties. Cyclic derivatives of amino acids can also be used. Cyclization can be obtained through amide bond formation, e.g., by incorporating Glu, Asp, Lys, Orn, di-amino butyric (Dab) acid, di-aminopropionic (Dap) acid at various positions is the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COON)—C(R)H—NH₂, wherein n=1-4, and further wherein R is any natural or non-natural side chain of an amino acid. Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Orn, Dab or Dap, Peptide bonds (—CO—NH—) within the peptide may be substituted by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom. These modifications can occur at any of the bonds along the peptide chain and even at several (2-3) at the same time. Natural aromatic amino acids, Trp, Tyr and Phe, may be substituted far synthetic port-natural acid such as TIC, naphthylelanine (Nol), ring-methylated derivatives of Phe, halogenated derivatives of Phe or o-methyl Tyr.

Display Libraries

According to still another aspect of the present invention there is provided a display library comprising a plurality of display vehicles (such as phages, viruses or bacteria) each displaying at least 5-10 or 15-20 consecutive amino acids derived from a polypeptide comprising contiguous amino acids having at least 80%, more preferably at least 90%, more preferably 95% or more homology (similar+identical amino acids) to positions 44 to 73 of SEQ ID NO:4.

According to a preferred embodiment of this aspect of the present invention substantially every 5-10 or 15-20 consecutive amino acids derived from a polypeptide comprising contiguous amino acids having at least 80%, preferably at least 90%, more preferably 95% or more homology (similar+identical amino acids) to positions 44 to 73 of SEQ ID NO:4 are displayed by at least one at the plurality of display vehicles, so as to provide a highly representative library. Preferably, the consecutive amino acids or amino acid analogs of the peptide or peptide analog according to this aspect of the present invention are derived from SEQ ID NO:4, preferably from the contiguous amino acids at positions 44-73 of SEQ ID NO:4.

Methods of constructing display libraries are well known in the art, such methods are described, for example, in Young A C, et al., “The three-dimensional structures of a polysaccharide binding antibody to Cryptococcus neoformans and its complex with a peptide from a phage display library: implications for the identification of peptide mimotopes” J Mol Biol Dec. 12, 1997; 274: 622-34; Giebel L B. et al. “Screening of cyclic peptide phage libraries identifies ligands that bind streptavidin with high affinities” Biochemistry Nov. 28, 1995; 34 (47): 15430-5; Davies E L et al., “Selection of specific phage-display antibodies using libraries derived from chicken immunoglobulin genes” J Immunol Methods Oct. 12, 1995; 186(1): 125-35; Jones C et al. “Current trends in molecular recognition and bioseparation” J Chromatogr A Jul. 14, 1995; 707(1): 3-22; Deng S J et al. “Basis for selection of improved carbohydrate-binding single-chain antibodies from synthetic gene libraries” Proc Natl Acad Sci USA May 23, 1995; 92(11): 4992-6; and Deng S J et al. “Selection of antibody single-chain variable fragments with improved carbohydrate bidding by phage display” J Biol Chem Apr. 1, 1994; 269(13): 9533-8, which are incorporated herein by reference. Display libraries according to this aspect of the present invention can be used to identify and isolate polypeptides which are capable of up- or down-regulating IL-1 activity.

Antibodies

According to still another aspect of the present invention there is provided an antibody comprising an immunoglobulin specifically recognizing and binding a polypeptide at least 80%, at least 85%, at least 90%, at least 95%, or 100% identical or homologous (identical+similar amino acids) to SEQ ID NOs: 2 or 4. According to a preferred embodiment of this aspect of the present invention the antibody specifically recognizes and binds to a polypeptide comprising contiguous amino acids having at least 80%, preferably at least 90%, more preferably 95% or more homology (similar+identical amino acids) to positions 44 to 73 set forth in SEQ ID NO:4.

The present invention can utilize serum immunoglobulins, polyclonal antibodies or fragments thereof, (i.e., immunoreactive derivative of an antibody), or monoclonal antibodies or fragments thereof, either produced by the modification of whole antibodies or those synthesized de novo using recombinant DNA methodologies. Monoclonal antibodies or purified fragments of the monoclonal antibodies having at least a portion of an antigen bidding region, including such as Fab, F(ab′)₂, Fv, scFv and the like (Harlow and Lane, 1988 Antibody, Cold Spring Harbor); single chain antibodies (U.S. Pat. No. 4,946,778); chimeric or humanized antibodies and complementarily determining regions (CDR) may be prepared by conventional procedures. These functional fragments of antibodies are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) complementarity-determining region (CDR) peptides (“minimal recognition units”) which can be obtained by constructing genes encoding the CDR of an antibody of interest, for example, by using the polymerase chain reaction to synthesize the variable region from RNA of antibody-producing cells. See, for example, Larrick and Fry [Methods, 2: 106-10 (1991)].

Purification of these serum immunoglobulins antibodies or fragments can be accomplished by a variety of methods known to those skilled in the art including, precipitation by ammonium sulfate or sodium sulfate followed by dialysis against saline, ion exchange chromatography, affinity or immunoaffinity chromatography as well as gel filtration, zone electrophoresis, etc. (Goding in, Monoclonal Antibodies: Principles and Practice, 2nd ed., pp. 104-126, 1986, Orlando, Fla., Academic Press). Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains. Additional classes include IgD, IgE, IgA, IgM and related proteins.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background. For example, more preferably the antibodies bind specifically to CGEN-R1 specific epitope(s) but do not bind to epitopes of known IL-1Ra proteins or variants (and/or bind at a much lower level, preferably being less than about half the level of binding to CGEN-R1 specific epitope(s)).

Monoclonal Antibodies

Methods for the generation and selection of monoclonal antibodies are well known in the art, as summarized for example in reviews such as Tramontano and Schloeder, Methods in Enzymology 178, 551-568, 1989. A recombinant or synthetic IL-1Ra or a portion thereof of the present invention may be used to generate antibodies in vitro. More preferably, the recombinant or synthetic IL-1Ra of the present invention is used to elicit antibodies in vivo. In general, a suitable host animal is immunized with the recombinant or synthetic IL-1Ra of the present invention or a portion thereof including at least one continuous or discontinuous epitope. Advantageously, the animal host used is a mouse of an inbred strain. Animals are typically immunized with a mixture comprising a solution of the recombinant or synthetic IL-1Ra of the present invention or portion thereof in a physiologically acceptable vehicle, and any suitable adjuvant, which achieves as enhanced immune response to the immunogen. By way of example, the primary immunization conveniently may be accomplished with a mixture of a solution of the recombinant or synthetic IL-1Ra of the present invention or a portion thereof and Freund's complete adjuvant, said mixture being prepared in the form of a water-in-oil emulsion. Typically the immunization may be administered to the animals intramuscularly, intradermally, subcutaneously, intraperitoneally, into the footpads, or by any appropriate route of administration. The immunization schedule of the immunogen may be adapted as required, but customarily involves several subsequent or secondary immunizations using a milder adjuvant such as Freund's incomplete adjuvant. Antibody titers and specificity of binding can be determined during the immunization schedule by any convenient method including by way of example radioimmunoassay, or enzyme linked immunosorbant assay, which is known as the ELISA assay. When suitable antibody titers are achieved, antibody producing lymphocytes from the immunized animals are obtained, and these are cultured, selected and closed, as is known in the art. Typically, lymphocytes may be obtained in large numbers from the spleens of immunized animals, but they may also be retrieved from the circulation, the lymph nodes or other lymphoid organs. Lymphocyte are then fused with any suitable myeloma cell line, to yield hybridomas, as is well known in the art. Alternatively, lymphocytes may also be stimulated to grow in culture; and may be immortalized by methods known in the art including the exposure of these lymphocytes to a virus; a chemical or a nucleic acid such as an oncogene, according to established protocols. After fusion, the hybridomas-ate cultured under suitable culture conditions, for example in multiwell plates, and the culture supernatants are screened to identify cultures containing antibodies that recognize the hapten of choice. Hybridomas that secrete antibodies that recognize the recombinant or synthetic IL-1Ra of the present invention are cloned by limiting dilution and expanded, under appropriate culture conditions. Monoclonal antibodies are purified and characterized in terms of immunoglobulin type and binding affinity.

Humanized Antibodies

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321: 522-525 (1986); Riechmann et al., Nature, 332: 323-329 (1988); and Presta, Curr. Op. Struct. Biol., 2: 593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321: 522-525 (1986); Riechmann et al., Nature 332: 323-327 (1988); Verhoeyen et al., Science, 239: 1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227: 381 (1991); Marks et al., J. Mol. Biol., 222: 581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boemer et al., J. Immunol., 147(1): 86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Pharmaceutical Composition for Regulation of IL-1R Activity

According to yet another aspect the present invention provides a pharmaceutical composition comprising, as an active ingredient, a CGEN-R1 agent for regulating an IL-1 activity in vivo or in vitro. The following embodiments of the present invention are directed at intervention with IL-1R activity and therefore with IL-1 signaling.

According to yet another aspect the present invention provides a method of regulating an endogenous protein affecting IL-1 receptor activity in vivo or in vitro.

According to one embodiment, the method according to this aspect of the present invention comprises the step of administering an agent for regulating the endogenous protein activity in vivo, the endogenous protein being at least 80%, at least 85%, at least 90%, at least 95%, or 100% homologous (identical+similar amino acids) to SEQ ID NOs: 2 or 4.

According to one preferred embodiment, the method comprises the step of administering an agent for regulating the endogenous protein activity in vivo, the endogenous protein comprising contiguous amino acids having at least 80%, preferably at least 90%, more preferably 95% or more homology (similar+identical amino acids) to positions 44 to 73 of SEQ ID NO:4.

An agent which can be used according to the present invention to upregulate the activity of the endogenous protein can include, for example, an expressible sense polynucleotide comprising a nucleotide sequence selected from the group consisting of SEQ ID NOs: 1, 3, 5, or 6.

According to one preferred embodiment, the agent used according to the present invention to upregulate the activity of the endogenous protein include an expressible sense polynucleotide encoding a polypeptide comprising contiguous amino acids having at least 80%, preferably at least 90%, more preferably 95% or more homology (similar+identical amino acids) to positions 44 to 73 of SEQ ID NO:4.

An agent which can be used according to the present invention to down-regulate the activity of the endogenous protein can include, for example, an expressible antisense polynucleotide comprising a nucleotide sequence complement to a sequence selected from the group consisting of SEQ ID NOs: 1, 3, 5, or 6.

According to one preferred embodiment, the agent used according to the present invention to downregulate the activity of the endogenous protein include an expressible antisense polynucleotide comprising contiguous nucleotides having the sequence of positions 130-219 of SEQ ID NO:3 or 6.

Alternatively, an agent which can be used according to the present invention to downregulate the activity of the endogenous protein can include, for example, an antisense oligonucleotide or ribozyme which includes a polynucleotide or a polynucleotide analog of at least 10 bases, preferably between 10 and 15, more preferably between 15 and 20 bases, most preferably, at least 17-40 bases which is hybridizable in vivo, under physiological conditions, with a portion of a polynucleotide strand encoding a polypeptide at least 80%, preferably at least 85%, more preferably at least 90% or more, most preferably at least 95% or more homologous to SEQ ID NOs: 2 or 4.

According to one currently preferred embodiment, the polynucleotide is hybridizable in vivo, under physiological conditions, with a portion of a polynucleotide strand encoding a polypeptide comprising contiguous amino acids having at least 80%, preferably at least 90%, more preferably 95% or more homology (similar+identical amino acids) to positions 44 to 73 of SEQ ID NO:4.

Still alternatively, an agent which can be used according to the present invention to downregulate the activity of the endogenous protein can include, for example, a peptide or a peptide analog representing a stretch of at least 6-10, 10-15, or 15-20 consecutive amino acids or analogs thereof derived from a polypeptide at least 80%, at least 85%, at least 90%, at least 95%, or 100% homologous (identical+similar amino acids) to SEQ ID NOs: 2 or 4.

According to one currently preferred embodiment the peptide or peptide analog downregulating the activity of the endogenous protein is derived from a polypeptide having at least 80%, preferably at least 90%, more preferably 95% or more homology (similar+identical amino acids) to positions 44 to 73 of SEQ ID NO:4.

Peptides or peptide analogs containing the interacting IL-1Ra-like domain according to the present invention will compete by protein interactions to form protein complexes with IL-1R, inhibiting or accelerating the pathways in which IL-1 is involved. Such peptides or peptide analogs may optionally comprise and/or be derived from the tail and/or bride portions of CGEN-R1 as previously described.

The following biochemical and molecular systems are known for the characterization and identification of protein-protein interaction and peptides as substrates, through peptide analysis, which systems can be used to identify inhibitory peptide sequences. One such system employs introduction of a genetic material encoding a functional protein or a mutated form of the protein, including amino acid deletions and substitutions, into cells. This system can be used to identify functional domains of the protein by the analysis of its activity and the activity of its derived mutants in the cells. Another such system employs the introduction of small encoding fragments of a gene into cells, e.g., by means of a display library or a directional randomly primed cDNA library comprising fragments of the gene, and analyzing the activity of the endogenous protein in their presence (see, for example, Gudkov et al. (1993) “Isolation of genetic suppressor elements, including resistance to topoisomerase II interactive cytotoxic drugs, from human topoisomerase II cDNA” Proc. Natl. Acad. Sci. USA 90: 3231-3236; Gudkov and Robinson (1997) “Isolation of genetic suppressor elements (GSEs) from random fragment cDNA libraries in retroviral vectors” Methods Mol Biol 69; 221-240; and Pestov et al. (1999) “Flow Cytometric Analysis of the cell cycle in transfected cells without cell fixation” Bio Techniques 26: 102-106). Yet an additional system is realized by screening expression libraries with peptide domains, as exemplified, for example, by Yamabhai et al. (1998 “Intersectin, a Novel Adaptor Protein with Two Eps 15 Homology and Five Src Homology 3 Domains”. J Biol Chem 273: 31401-31407). In yet another such system overlapping synthetic peptides derived from specific gene products are used to study and affect in vivo and in vitro protein-protein interactions. For example, synthetic overlapping peptides derived from the HIV-1 gene (20-30 amino acids) were assayed for different viral activities (Baraz et al. (1998) “Human immunodeficiency virus type 1 Vif derived peptides inhibit the viral protease and arrest virus production” FEBS Letters 441: 419-426) and were found to inhibit purified viral protease activity; bind to the viral protease; inhibit the Gag-Pol polyprotein cleavage; and inhibit mature virus production in human cells.

Other agents according to the present invention may optionally include an antibody capable of specifically recognizing an epitope of CGEN-R1, wherein such an epitope preferably comprises a tail and/or bridge portion as previously described. Such an antibody may have a therapeutic utility in blocking or decreasing the activity of the IL-1Ra splice variant protein in pathological conditions where beneficial effect can be achieved by such a decrease. The antibody employed is preferably a humanized monoclonal antibody, produced by known globulin-gene library methods. The antibody is administered typically as a sterile solution by IV injection, although other parenteral routes may be suitable. Typically, the antibody is administered in an amount between about 1-15 mg/kg body weight of the subject. Treatment is continued, e.g., with dosing every 1-7 days, until a therapeutic improvement is seen.

Transgenic Animals or Cell Lines

The present invention has the potential to provide transgenic gene and polymorphic gene animal and cellular (cell lines) models as well as knock-out and knock-in models. These models may be constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson (1991) Methods in Enzymology, 194: 251-270; Capecchi (1989) Science 244: 1288-1292; Davies et al. (1992) Nucleic Acids Research, 20 (11) 2693-2698; Dickinson et al. (1993) Human Molecular Genetics, 2(8): 1299-1302; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al. (1991) Genomics, 9: 7414 750 1991; Jakobovits et al. (1993) Nature, 362: 255-261; Lamb et al. (1993) Nature Genetics, 5: 22-29; Pearson and Choi, (1993) Proc. Natl. Acad. Sci. USA 90: 10578-82; Rothstein, (1991) Methods in Enzymology, 194: 281-301; Schedl et al. (1993) Nature, 362: 258-261; Strauss et al. (1993) Science, 259; 1904-1907. Further, patent applications WO 94/23049, WO 93/14200, WO 94/06408, WO 94/28123 also provide information.

All such transgenic gene and polymorphic gene animal and cellular (cell lines) models and knockout or knock-in models derived from claimed embodiments of the present invention, constitute preferred embodiments of the present invention.

Gene Therapy

Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a ligand, hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (i) ex vivo and (ii) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed, and then the modified cells are expanded in culture and returned to the host/patient. These genetically reimplanted cells have been shown to express the transfected genetic material in situ.

In in vivo gene therapy, target cells are not removed from the subject. Rather, the genetic material to be transferred is introduced into the cells of the recipient organism in situ, that is within the recipient. In an alternative embodiment, if the host gene is defective, the gene is repaired in situ. These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequences which work contiguously with the coding sequence (in cis) to change the basal transcription level dictated by the promoter. The expression vehicle can also include a selection gene as described hereinbelow.

Vectors Useful in Gene Therapy

As described herein above, vectors can be introduced into host cells or tissues by any one of a variety of known methods within the art.

Introduction of nucleic acids by infection offers several advantages over the other listed methods, specifically higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Features that limit expression to particular cell type can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The natural specificity of viral vectors is utilized to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles, which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, they do not have to be administered locally at the diseased site. However, when local administration can provide a quicker and more effective treatment, administration can also be performed by, for example, intravenous or subcutaneous injection into the subject. Injection of the viral vectors into a spinal fluid can also be used as a mode of administration. Following injection, the viral vectors will circulate until they recognize cells with appropriate target specificity for infection.

According to an alternative embodiment, the nucleic acid constructs according to the present invention further include a positive and a negative selection markers and may therefore be employed for selecting for homologous recombination events, including, but not limited to, homologous recombination employed in knock-in and knockout procedures. One ordinarily skilled in the art can readily design a knockout or knock-in constructs including both positive and negative selection genes for efficiently selecting transfected embryonic stem cells that underwent a homologous recombination event with the construct. Such cells can be introduced into developing embryos to generate chimeras, the offspring thereof can be tested for carrying the knockout or knock-in constructs. Knockout and/or knock-in constructs according to the present invention can be used to further investigate the functionality of CGEN-R1. Such constructs can also be used in somatic and/or germ cells gene therapy to increase/decrease the activity of IL-1, thus regulating IL-1 related inflammatory and immune responses. Further detail relating to the construction and use of knockout and knock-in constructs can be found in Fukushige, S. and Ikeda, J. E. (1996) DNA Res 3: 73-50; Bedell, M. A. et al. (1997) Genes and Development 11: 1-11; Bermingham, J. J. et al. (1996) Genes Dev 10: 1751-62, which are incorporated herein by reference.

Antisense Polynucleotides

According to some embodiments the present invention provides antisense polynucleotides useful for regulation of the expression of CGEM-R1, affecting the IL-1/IL-1 receptor interactions. In diseases or disorders related to IL-1 receptors, namely in inflammatory and immunogenic process, therapeutic effect is typically reached by employing IL-1 receptor antagonists. However, when the native activity of IL-1 is redundant, or in pathologies where enhanced IL-1 activity is desired, preferred therapeutic effect may be achieved by blocking the activity of IL-1 receptor antagonists. For example, when enhanced immune response is required, CGEN-R1 antisense polynucleotides may be used as anticancer and antiviral agents, in wound healing, as analgesic for enhancing the release of cytoplasmic granule-associated elastase from human neutrophils and the like.

Thus, according to an additional embodiment of the present invention there is provided an antisense oligonucleotide comprising a polynucleotide or a polynucleotide analog of at least 10 bases, preferably between 10 and 15, more preferably between 5 and 20 bases, most preferably, at least 17-40 bases being hybridizable in vivo, under physiological conditions, with a portion of a polynucleotide strand encoding a polypeptide at least 80%, at least 85%, at least 90% or more, at least 95%, or 100% homologous (similar+identical amino acids) to SEQ ID NOs: 2 or 4.

According to one preferred embodiment, the present invention provides an antisense oligonucleotide comprising a polynucleotide or a polynucleotide analog of at least 10 bases, preferably between 10 and 15, more preferably between 5 and 20 bases, most preferably, at least 17-40 bases being hybridizable in vivo, under physiological conditions, with a portion of a polynucleotide strand encoding contiguous amino acids having at least 80%, preferably at least 90%, more preferably 95% or more homology (similar+identical amino acids) to positions 44 to 73 of SEQ ID NO:4.

Such antisense oligonucleotides can be used to down regulate expression as further detailed hereinunder. Such an antisense oligonucleotide is readily synthesizable using solid phase oligonucleotide synthesis.

The ability of chemically synthesizing oligonucleotides and analogs thereof having a selected predetermined sequence offers means for down-modulating gene expression, as well as for altering or restoring the expression of a given gene. Three types of gene expression modulation strategies may be considered.

At the transcription level, antisense or sense oligonucleotides or analogs that bind to the genomic DNA by strand displacement or the formation of a triple helix, may prevent transcription. At the transcript level, antisense oligonucleotides or analogs that bind target mRNA molecules lead to the enzymatic cleavage of the hybrid by intracellular RNaseH. In this case, by hybridizing to the targeted mRNA, the oligonucleotides or oligonucleotide analogs provide a duplex hybrid recognized and destroyed by the RNaseH enzyme. Alternatively, such hybrid formation may lead to interference with correct splicing, which may results in down regulation of the gene expression but may also modulate the expression of novel splice variants.

At the translation level, antisense oligonucleotides or analogs that bind target mRNA molecules prevent, by steric hindrance binding of essential translation factors (ribosomes), to the target mRNA a phenomenon known in the art as hybridization arrest, disabling the translation of such mRNAs.

Thus, antisense sequences, which as described hereinabove may arrest or modify the expression of any endogenous and/or exogenous gene depending on their specific sequence, are subjects for the development of a new pharmacological tool.

For example, several antisense oligonucleotides have been shown to arrest hematopoietic cell proliferation (Szczylik et al., 1991), growth (Calabretta et al.; 1941), entry-into the S phase of the cell cycle (Heikhila et al., 1987), reduced survival (Reed et al., 1990) and prevent receptor mediated responses (Burch and Mahan, 1991). In addition, radio labeled or otherwise labeled antisense oligonucleotides can be used as diagnostic tools, in vitro as well as in vivo, for example for imaging a specific mRNA, for monitoring antisense chemotherapy, and for protein imaging.

For efficient in vivo application of antisense oligonucleotides or analogs, the oligonucleotides or analogs must fulfill the following requirements (i) sufficient specificity in binding to the target sequence; (ii) solubility in water; (iii) stability against intra- and extracellular nucleases; (iv) capability of penetration through the cell membrane; and (v) when used to treat an organism, low toxicity.

Unmodified oligonucleotides are typically impractical for use as antisense sequences since they have short in vivo half-lives, during which they are degraded rapidly by nucleases. Furthermore, they are difficult to prepare in more than milligram quantities. In addition, such oligonucleotides have poor penetration into or through cell membranes.

Thus it is apparent that in order to meet all the above listed requirements, oligonucleotide analogs need to be devised in a suitable manner, as described herein below.

Oligonucleotide Analogs

Oligonucleotide analogs are produced in order to improve half-life as well as membrane penetration. Oligonucleotides can be modified either in the base, the sugar or the phosphate moiety. These modifications include, for example, the use of methylphosphonates, monothiophosphates, dithiophosphates, phosphoramidates, phosphate esters, bridged phosphorothioates, bridged phosphoramidates, bridged methylenephosphonates, dephospho internucleotide analogs with siloxane bridges, carbonate brides, carboxymethyl ester bridges, carbonate bridges, carboxymethyl ester bridges; acetamide bridges, carbonate bridges, thioether bridges, sulfoxy bridges, sulfono bridges, various “plastic” DNAs, α-anomeric bridges and borane derivatives.

International Patent Application WO 89/12060 discloses various building blocks for synthesizing oligonucleotide analogs, as well as oligonucleotide analogs formed by joining such building blocks in a defined sequence. The building blocks may be either “rigid” (i.e., containing a ring structure) or “flexible” (i.e., lacking or ring structure). In both cases, the building blocks contain a hydroxy group and a mercapto group, through which the building blocks are said to join to form oligonucleotide analogs. The linking moiety in the oligonucleotide analogs is selected from the group consisting of sulfide (—S—), sulfoxide (—SO—), and sulfone (—SO₂—).

International Patent Application WO 92/20702 describe an acyclic oligonucleotide which includes a peptide backbone on which any selected chemical nucleobases or analogs are stringed and serve a coding characters as they do in natural DNA or RNA. These new compounds, known as peptide nucleic acids (PNAs), are not only more stable in cells than their natural counterparts, but also bind natural DNA and RNA, 50 to 100 times more tightly than the natural nucleic acids cling to each other. PNA oligomers can be synthesized from the four protected monomers containing thymine, cytosine, adenine and guanine by Merrifield solid-phase peptide synthesis. In order to increase solubility in water and to prevent aggregation, a lysine amide group is placed at the C-terminal region.

Thus, in one preferred aspect antisense technology requires pairing of messenger RNA with an oligonucleotide to form a double helix that inhibits translation. The concept of antisense-mediated therapy was already introduced in 1978 for cancer therapy. This approach was based on certain genes that are crucial in cell division and growth of cancer cell. Synthetic fragments of genetic substance DNA can achieve this goal. Such molecules bind to the targeted gene molecules in RNA of tumor cells, thereby inhibiting the translation of the gates and resulting in dysfunctional growth of these cells. Other mechanisms have also been proposed. These strategies have been used with some success is treatment of cancers, as well of other illnesses, including viral and other infectious diseases. Antisense oligonucleotides are typically synthesized in lengths of 13-30 nucleotides. The life span of oligonucleotide molecules in blood is rather short. Thus, they have to be chemically modified to prevent destruction by ubiquitous nucleases present in the body. Phosphorothioates are very widely used modification in antisense oligonucleotide ongoing clinical trials. A new generation of antisense molecules consists of hybrid antisense oligonucleotide with a central portion of synthetic DNA while four bases on each end have been modified with 2′O-methyl ribose to resemble RNA. In pre-clinical studies in laboratory animals, such compounds have demonstrated greater stability to metabolism in body tissues and an improved safety profile when compared with the first-generation unmodified phosphorothioate (Hybridon Inc. news). Dozens of other nucleotide analogs have also been tested in antisense technology.

RNA oligonucleotides may also be used for antisense inhibition as they form a stable RNA-RNA duplex with the target, suggesting efficient inhibition. However, due to their low stability RNA oligonucleotides are typically expressed inside the cells using vectors designed for this purpose. This approach is favored when attempting to target a mRNA that encodes an abundant and long-lived protein.

Recent scientific publications have validated the efficacy of antisense compounds in animal models of hepatitis, cancers, coronary artery restenosis and other diseases. The first antisense drug was recently approved by the FDA. This drug Fomivirsen, developed by Isis, is indicated for local treatment of cytomegalovirus in patients with AIDS who are intolerant of or have a contraindication to other treatments for CMV retinitis or who were insufficiently responsive to previous treatments for CMV retinitis (Pharmacotherapy News Network).

Several antisense compounds are now in clinical trials in the United States. These include locally administered antivirals, systemic cancer therapeutics. Antisense therapeutics has the potential to treat many life-threatening diseases with a number of advantages over traditional drugs. Traditional drugs intervene after a disease-causing protein is formed. Antisense therapeutics, however, block mRNA transcription/translation and intervene before a protein is formed, and since antisense therapeutics target only one specific mRNA, they should be more effective with fewer side effects than current protein-inhibiting therapy.

A second option for disrupting gene expression at the level of transcription uses synthetic oligonucleotides capable of hybridizing with double stranded DNA. A triple helix is formed. Such oligonucleotides may prevent binding of transcription factors to the gene's promoter and therefore inhibit transcription. Alternatively they may prevent duplex unwinding and, therefore, transcription of genes within the triple helical structure.

Thus, according to a further aspect of the present invention there is provided a pharmaceutical composition comprising the antisense oligonucleotide described herein and a pharmaceutically acceptable carries. The pharmaceutically acceptable carrier can be, for example, a liposome loaded with the antisense oligonucleotide. Formulations for topical administration may include, but are not limited to, lotions, ointments, gels, creams, suppositories, drops, liquids, sprays and powders.

Conventional pharmaceutical carriers, aqueous, powder or oily bases, thickeners and the like may be necessary or desirable. Compositions for oral administration include powders or granules, suspensions or solutions in water or non-aqueous media, sachets, capsules or tablets. Thickeners, diluents, flavorings, dispersing aids, emulsifiers or binders may be desirable. Formulations for parenteral administration may include but ate not limited to, sterile aqueous solutions which tray also contain buffers, diluents and other suitable additives.

According to still a further aspect of the present invention there is provided a ribozyme comprising the antisense oligonucleotide described herein and a ribozyme sequence fused thereto. Such a ribozyme is readily synthesizable using solid phase oligonucleotide synthesis.

Ribozymes are being increasingly used for the sequence-specific inhibition of gene expression by the cleavage of mRNAs encoding proteins of interest. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders. Most notably, several ribozyme gene therapy protocols for HIV patients are already in Phase 1 trials. More recently, ribozymes have been used for transgenic animal research, gene target validation and pathway elucidation Several ribozymes are in various stages of clinical trials. ANGIOZYME was the first chemically synthesized ribozyme to be studied in human clinical orals. ANGIOZYME specifically inhibits formation of Vascular Endothelial Growth Factor receptor (VEGF-R), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated-WEB home page).

Diagnostic Applications

The level of CGEN-R1 within a cell or a tissue is expected to have diagnostic value, for example by diagnosing cancer. For example, it is taught that diagnosis of endometrial cancer can be made by measuring the amount of intracellular IL-1Ra present in endometrial cells from a patient suspected of having said cancer, and comparing the amount to that present in normal endothelial cells is disclosed in U.S. Pat. No. 5,840,496. Methods for diagnosing diseases resulting from undesirable cell adhesion of IL-1 receptor positive cells to biological material, particular to endothelial cells, or autoimmune related diseases, or IL-1 dependent cancer by measuring the amount of intracellular IL-1Ra present are disclosed in U.S. Pat. No. 5,814,469; such methods may also be suitable for CGEN-R1 according to the present invention. Other diagnostic assays and methods that may optionally be implemented with the variants according to the present invention are described in U.S. Pat. No. 5,872,095. All patents are hereby incorporated by reference as if fully set forth herein.

Hybridization Assays

Detection of a nucleic acid of interest in a biological sample may optionally be effected by hybridization-based assays using an oligonucleotide probe.

Hybridization based assays which allow the detection of a variant of interest (i.e., DNA or RNA) in a biological sample rely on the use of oligonucleotide which can be 10, 15, 20, or 30 to 100 nucleotides long preferably from 10 to 50, more preferably from 40 to 50 nucleotides.

Hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif] can be attached to the oligonucleotides.

Hybridization assays (or assays with a hybridization component) include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10: 71-75). Other detection methods include kits containing probes on a dipstick setup and the like.

Although the present invention is not specifically dependent on the use of a label for the detection of a particular nucleic acid sequence, such a label might be beneficial, by increasing the sensitivity of the detection.

Furthermore, it enables automation. Probes can be labeled according to numerous well known methods (Sambrook et al., 1989, supra). Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ATP and polynucleotide kinase, using the Klenow fragment of Pol I of E coli in the presence of radioactive dNTP (i.e. uniformly labeled DNA probe using random oligonucleotide primers in low-melt gels), using the SP6/T7 system to transcribe a DNA segment in the presence of one or more radioactive NTP, and the like.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Probes of the invention can be utilized with naturally occurring sugar-phosphate backbones as well as modified backbones including phosphorothioates, dithionates, alkyl phosphonates and a-nucleotides and the like. Modified sugar-phosphate backbones are generally taught by Miller, 1988, Ann. Reports Med. Chem. 23: 295 and Moran et al., 1987, Nucleic acid molecule. Acids Res., 14: 5019. Probes of the invention can be constructed of either ribonucleic acid (RNA) or deoxyribonucleic acid (DNA), and preferably of DNA.

Optionally and preferably, such probes are constructed according to the nucleotide sequences corresponding to the tail and/or bridge portions of CGEN-R1 according to the present invention, as described above.

NAT Assays

Detection of a nucleic acid of interest in a biological sample may also optionally be effected by NAT-based assays, which involve nucleic acid amplification technology, such as PCR for example (or variations thereof such as real-time PCR for example).

Amplification of a selected, or target, nucleic acid sequence may be carried out by a number of suitable methods. See generally Kwoh et al., 1990, Am. Biotechnol. Lab. 8: 14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6: 1197-1202; Malek et al., 1994, Methods Mol. Biol., 28: 253-260; and Sambrook et al., 1989, supra).

Polymerase chain reaction (PCR) is carried out in accordance with known techniques, as described for example, in U.S. pat. Nos. 4,683,195; 47683,202; 4,800,159; and 4,965,188 (the disclosures of all three U.S. patents are incorporated herein by reference). In general, PCR involves a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review of PCR techniques, see PCR Protocols, A Guide to Methods and Amplifications, Michael et al. Eds, Acad. Press, 1990.

Optionally and preferably, such primers are constructed according to the nucleotide sequences corresponding to the tail and/or bridge portions of CGEN-R1 according to the present invention, as described above.

Ligase chain reaction (LCR) is carried out in accordance with known techniques (Weiss, 1991, Science 254: 1292). Adaptation of the protocol to meet the desired needs can be carried out by a person of ordinary skill. Strand displacement amplification (SDA) is also carried out in accordance with known techniques or adaptations thereof to meet the 1 5-particular needs (Walker et al., 1992, Proc. Natl. Acad. Sci. USA 89: 392-396; and ibid., 1992, Nucleic Acids Res. 20: 1691-1696).

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. Of course, it will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. In general, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

Diagnostic Applications of Antibodies

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

Optionally and preferably, such antibodies are prepared according to the amino acid sequences corresponding to the tail and/or bridge portions of CGEN-R1 according to the present invention, as described above.

The detection and/or quantifying of a marker can be made using any of a number of well recognized immunological binding assays (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. For a review of the general immunoassays, see also, Methods in Cell Biology: Antibodies in Cell Biology, volume 37 (Asai, ed. 1993); Basic and Clinical Immunology (Stites & Terr, eds., 7th ed. 1991).

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a substrate as described above. The sample is preferably a biological fluid sample taken from a subject. Examples of biological fluid samples include blood, serum, urine, prostatic fluid, seminal fluid, semen, seminal plasma and prostate tissue (e.g., epithelial tissue, including extracts thereof) as well as amniotic fluid. In a preferred embodiment, the biological fluid comprises seminal plasma. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The principles of the invention, disclosing novel IL-1Ra splice variants, polynucleotides encoding same, methods of productions, pharmaceutical composition comprising same and methods for use may be better understood with reference to the following non-limiting examples.

### EXAMPLES

### Example 1

### Identification of CGEN-R1

The mRNAs sequences of the known IL-1Ra (SEQ ID NO:7) and its variants (SEQ ID NO:14; SEQ ID NO:12; SEQ ID NO:13), were used for screening an EST database for novel splice variants using proprietary algorithm for clustering and assembly of nucleic acid sequences (the method for mRNA clustering and assembly used described in U.S. patent application Ser. No. 09/133,987. The screening and annotation method described-in U.S. patent application Ser. Nos. 10/426,002, 10/242,799 assigned to the assignee of the present invention). Three types of EST clones were revealed:
(i) An EST clone containing part of exon 1 and exon 2 joined to a unique sequence, derived from an intron 2 of the wild type IL-1Ra (SEQ ID NO:7). This EST was identified in a human cDNA library NIH_MGC_{—}120 (Pooled Pancreas and Spleen, Accession number B1836973).
(ii) Four EST clones with high homology to the above unique intron-derived sequence were found to be transcribed, with the transcript having no homology to known IL-1Ra sequences, in the following human cDNA libraries: NCI_CGAP_GU1 (2 pooled high-grade transitional cell tumors) cDNA library (Accession number: AW630035); NT0022 (Nervous Tumor) cDNA library (Accession number: BF365244); Stratagene liver (#937224) (Liver) cDNA library (Accession number: T71181) and HT0125 (Head and Neck tumor) cDNA library (Accession number: AW178803).
(iii) An EST clone originating from a human HT0366 (Head and Neck Tumor) cDNA library (Accession number BE706905) encoding a unique intron 2-derived sequence joined to exon 3 of the known IL-1Ra (SEQ ID NO:7).

All above ESTs and cDNA sequences support the retention of intron 2 of IL-1Ra (SEQ ID NO:7) in the coding sequence of the novel splice variant of IL-1Ra.

While reducing the present invention to practice these clones have been characterized as encoding previously unknown antagonist of the interleukin-1 receptor (IL-1Ra), which is referred to herein as CGEN-R1 (SEQ ID NO:2 or 4). This novel IL-1 receptor antagonist comprise as a C terminal portion a unique amino acid sequence sharing no homology to the known human IL-1Ra sequence of SEQ ID NO:8 (FIG. 1), nor to any other known IL-1 receptor antagonist or other protein in the public database.

### Example 2

### Expression of the Novel CGEN-R1 Splice Variant

The expression of the novel CGEN-R1 splice variant was examined by RT-PCR in the following tissues and conditions: Crohn's inflammatory colon tissue, colon, spleen, bone marrow, liver, thymus, pancreas, melanoma cell line.

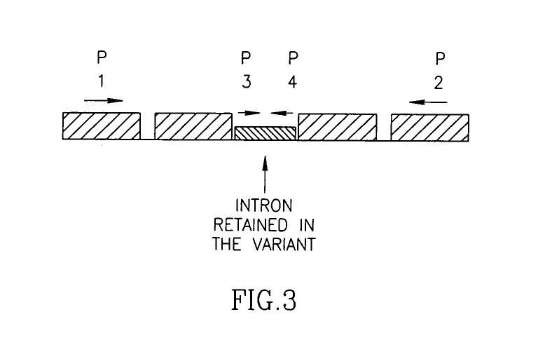

The following primers (schematically described in FIG. 3) were used for the RT-PCR:

| | |
|---|---|
| Primer 1: | |
| IL-1Ra Variant flanking exon F: | |
| CAGAGGCCTCCGCAGTCACC. | (SEQ ID NO:15) |
| Primer 2: | |
| IL-1Ra Variant flanking exon R: | |
| TGACGGGCTGGTCAGCTTCC. | (SEQ ID NO:16) |
| Primer 3: | |
| IL-1Ra Variant specific F: | |
| GGCAGCCTGAAGAGGGTGTGG. | (SEQ ID NO:17) |
| Primer 4: | |
| IL-1Ra Variant specific R: | |
| TCCCACTGAAGGGAAAGCTGAGG. | (SEQ ID NO:18) |

The RT PCR conditions were as follows: the reaction mixture contained in a final volume of 25 μl: 0.5 μl of specific primers, 25 μM; 1 μl of cDNA; 2.5 μl of 10× reaction buffer (Qiagen); Hotstar Taq polymerase (Qiagen) 0.5 μl; dNTPs (Takara) 2 μl of 5 mM each. The RT-PCR was performed with 1 cycle of 15 minutes at 95° C., followed by 35 cycles including denaturation step of 30 seconds at 94° C.; annealing at 61° C. for 45 seconds and extension at 72° C. for 2 minutes. Final annealing step was performed at 72° C. for 10 minutes.

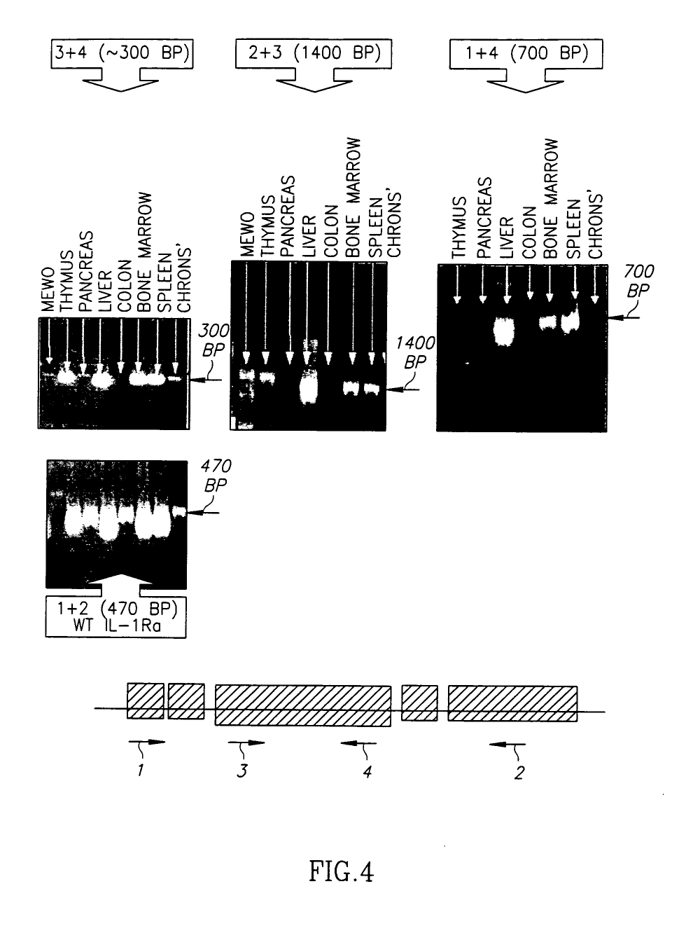

Primers 1 and 2 were used to detect transcripts of wild type IL-1Ra. Expression of the wild type IL-1Ra (a product of 470 bp, FIG. 4)) was detected in all the tissues examined. Using the specific primers for the novel splice variant CGEN-R1, primers 3 and 4 (product of 300 bp, FIG. 4), high transcript levels were found in intestinal tissues obtained in spleen, bone marrow, liver and thymus. Low transcript amounts were also found in pancreas, in a colon sample from a patient with Crohn's disease, and in a malignant melanoma cell line (MeWo). Transcripts of CGEN-R1 were not detected in normal colon tissues. RT-PCR reactions with primer 2 and 3 or 1 and 4 (products of 1400 bp and 700 bp, respectively; FIG. 4), also specific to the novel CGEN-R1 splice variant, revealed high transcript amount in liver tissue; somewhat lower amount was shown for spleen, bone marrow and thymus tissues and only small amount was detected in MeWo. In summary, the novel splice variant CGEN-R1 was detected in several human tissues, and was particularly highly expressed in liver tissues. This finding is in accordance with the high expression level found for wild type IL-1Ra in liver HepG2 cell line.

### Example 3

### Cloning of the Variant

mRNA from normal liver and spleen (sample # 081P0101A—from AMBION) was isolated and subjected to reverse transcription (RT) using random hexamer primer mix and Superscript™, followed by a treatment of RNAse I. The wild type IL-1Ra and the CGEN-R1 novel splice variant fragments for cloning were prepared by PCR amplification using TaKaRa Hot-Start Ex-Taq™ under the following conditions: 2.5 μl—Ex-Taq X10 buffer; 5 μl—cDNA; 2 μl—dNTPs (2.5 mM each); 0.5 μl—Ex-Taq enzyme; 14 μl—H₂O; and 0.5 μl—of each primer in a total reaction volume of 25 μl; with a reaction program of 5 minutes in 95° C.; 40 cycles of: 30 seconds at 94° C., 45 seconds at 68° C., 60 seconds at 72° C. and 10 minutes at 72° C.

The following primers, comprising specific sequences of the nucleotide sequence corresponding to the splice variant and/or the wild type, and Gateway™ BP recombination tails were used:

Primer 5:
IL-1Ra forward primer with a signal peptide

(SEQ ID NO:19)

5′GGGGACAAGTTTGTACAAAAAAGCAGGCTCCATGGAAATCTGCAGAGG
CCTCCG 3′.
Primer 6:
IL-1Ra forward primer without a signal peptide

(SEQ ID NO:20)

5′GGGGACAAGTTTGTACAAAAAAGCAGGCTCCATGACGATCTGCCGACC
CTCTGG 3′.
Primer 7:
CGEN-R1 novel splice variant CGR1 reverse primer

(SEQ ID NO:21)

5′GGGGACCACTTTGTACAAGAAAGCTGGGTAGACCCAACAAGGATTAGG
ACATTGCAC3′.
Primer 8:
wild type IL-1Ra reverse primer

(SEQ ID NO:22)

5′GGGGACCACTTTGTACAAGAAAGCTGGGTATCCCTGCAGTCCTT
GCCATGC3′.

Reaction with primer set 5+7 gave the CGEN-R1 novel splice variant with a signal peptide (385 bp).

Reaction with primer set 5+8 gave the wild type IL-1Ra with a signal peptide (646 bp).

Reaction with primer set 6+7 gave the CGEN-R1 novel splice variant without a signal peptide (322 bp).

Reaction with primer set 6+8 gave the wild type IL-1Ra without a signal peptide (583 bp).

PCR products were run in a 2% agarose gel, TBEX1 solution at 150V, and extracted from gel using QiaQuick™ gel extraction kit (Qiagen™). The extracted DNA products were sequenced by direct sequencing using Gateway primers (Forward—5′-GGGGACAAGTTTGTACAAAAAAGCAGGCT-3′ (SEQ ID NO:23) and Reverse—5′-GGGGACCACTTTGTACAAGAAAGCTGGG-3′ (SEQ ID NO:24). Error-free inserts were introduced into Gateway™ entry clone (Invitrogen™) by a BP clonase reaction (according to the manufacturer protocol), and DH5α competent bacteria were transfected with the resulted clones using the following protocol:

5 μl of each BP reaction product were mixed with freshly thawed 50 PI of competent DH5α cells. The mix was incubated on ice for 30 minutes and then exposed to Heat-Shock at 42° C. for 30 seconds. 450 μl of LB was added to each tube, and the tubes were incubated at 37° C. in a shaker for 1 hour.

From each transfection solution, both 50 μl and 150 μl were plated on selective LB plates containing 50 μg/ml Kanamycin. The plates were incubated at 37° C. overnight.

10 Colonies from each transcript clone that grew on the selective plates were taken for further analysis by re-plating on a selective plate and by PCR. PCR was performed using primers specific to the vector (pDONR), located upstream and downstream to the insert:

| | |
|---|---|
| pDONR-Forward: | |
| 5′-CGCGTTAACGCTAGCATGGAT-3′. | (SEQ ID NO:25) |
| pDONR-Reverse: | |
| 5′-CACAGAGTTTTAGAGACTACAAT-3′. | (SEQ ID NO:26) |

PCR products were extracted and sequenced as above, using the Gateway primers (SEQ ID NO:23 and SEQ ID NO:24).

Colonies containing an error free insert (no mutations within the ORF), were grown overnight in 2 ml of LB+50 ng Kanamycin at 37° C. Plasmids were obtained from bacterial colonies using Qiaprep™ spin miniprep kit (Qiagen). Plasmid's inserts were transferred into pDEST destination vectors (Gateway™—Invitrogen) according to the manufacturer protocol. Both constructs (wild type IL-1Ra and CGEN-R1) with a signal peptide were transferred into pDEST26™ (Mammalian expression vector), and both constructs without the signal peptides were transferred into pDEST17 (Bacterial expression vector carrying penta-His tag at its 5′end). Accurate cloning was verified by sequencing the clones' inserts.

### Example 4A

### Purification of His-tagged IL-1Ra Wild Type and CGEN-R1 Splice Variant (Without Signal Peptide) from E. Coli

Wild type IL-1Ra was expressed in E. Coli, and the expression level was optimized using an IPTG-inducing system. IPTG was added at concentrations of 0.1 mM, 0.5 mM and 1 mM for different incubation times.

The method used was as follows. Expression was induced by streaking transfected bacteria onto pre-warmed LB agar plates containing ampicillin (AMP) and chloramphenicol (CLAM) and grown overnight at 37° C. Thereafter, spools of bacteria were taken to inoculate 100 ml pre-warmed TB media containing AMP and CLAM and allowed to grow at 37° C. with vigorous aeration. At OD=˜0.7, flasks were immediately transferred to 4° C. and maintained overnight. The following day, 500 ml TB media (containing AMP and CLAM) was pre-warmed at 37° C. Twenty-five ml of the overnight starter was centrifuged for 5 min, supernatant removed and cells pelleted in 5 ml fresh TB media. This 5 ml cell suspension was taken to inoculate 500 ml TB and grown to OD=0.7-0.8. At this point, a 5 ml growth culture was removed and transferred to a 50 ml tube (=non-induced sample). To the remaining 495 ml TB, 0.495 ml 1 M IPTG (Roche) was added and induction was allowed to proceed for 3 hrs. After 3 hrs, cell aliquots were taken prior to centrifugation of the entire 500 ml induction.

Purification of Bacterial-Expressed IL-1Ra WT and CGEN-R1 Via Immobilized Metal Affinity Chromatography (IMAC)

Preparation of native extracts: Following IPTG inductions and centrifugation of cell pellet, cells were resuspended in 1/10 volume of a native lysis buffer to a final concentration of 50 mM NaHPO₄, 300 mM NaCl, 10 mM imidazole and protease inhibitors 1/50 (Roche). Following addition of Iysozme to 1 mg/ml, and incubation on ice for 30 min, the viscous cell suspension was taken for sonication using settings of 30 sec on/off, 5× with pulse at 3 setting. The subsequent clear cell suspension was centrifuged for 30 min at 10,000 g at 4° C. and the clarified supernatant taken for IMAC using Nickel agarose beads (Qiagen).

Preparation of denatured extracts: Following IPTG inductions and centrifugation of cell pellet, cells were resuspended in denaturing lysis buffer consisting of 8 M urea and 300 mM NaCl, 10 mM imidazole. Following extensive agitation for 30 min at room temperature, the solubilized material was centrifuged for 30 min at 10,000 g at 4° C. and the clarified supernatant taken for IMAC using Nickel agarose beads (Qiagen).

Purification of extracts by IMAC: Appropriate volumes of Nickel agarose were centrifuged to get rid of ethanol, the resin was resuspended gently with water and centrifuged (×2). After discarding the water, the resin was gently resuspended with clarified extracts and binding allowed to proceed at 4° C. for at least 60 min. Following binding, resin was washed with increasing concentrations of imidazole (20-500 mM) in respective lysis buffer to elute purified protein. Washing/elution was performed by either repeated rounds of centrifugation or after loading on disposable 5 ml polypropylene columns. Following the final elution with imidazole, resin was treated with 0.1 M EDTA to strip Nickel from the resin to assay for very strongly bound material.

Purification by Prospec Tany TechnoGene Ltd (Rehovot Israel)

The method involved solubilization of induced bacterial pellet under denaturing conditions, HPLC purification and re-folding to attain a biologically active protein. The method is proprietary to Prospec Tany TechnoGene Ltd (Rehovot Israel).

Antibody Preparation

Rabbits were immunized with a peptide which constitutes part of the CGEN-R1 unique tail (peptide sequence ═N′-GEWLPGKPMYVGITSLC-C′, SEQ ID NO:27), to produce a polyclonal antibody. The peptide corresponds to amino acid positions 44-60 of SEQ ID NO:4. The antibodies were prepared by Sigma Israel (Israel). Briefly, rabbits were injected according to the following schedule (Table I):

TABLE I
immunization schedule for polyclonal antibody production in rabbits

| Injection/bleeding | Day count |
|---|---|
| Pre immune bleed | 0 |
| Injection 1 (CFA) | 0 |
| Injection 2 (IFA) | 14 |
| Injection 3 (IFA) | 28 |
| Test bleed 1 | 35 |
| Injection 4 (IFA) | 42 |
| Test bleed 2 | 49 |
| Injection 5 (IFA) | 56 |
| Test bleed 3 | 63 |
| Injection 6 (IFA) | 70 |
| Bleed 4 (40 ml from each rabbit) | 77 |
| Bleed 5 (90 ml from 1561 and 75 ml from 1562) | Collected until next |
| | boost |
| Pre-boost test | 104 |
| Injection 7 (IFA) | 104 |
| Bleed 6 (40 ml from each rabbit) | 121 |

“IFA” refers to incomplete Freund's adjuvant, while “CFA” refers to complete Freund's adjuvant.

The antibodies in the above bleeds were first screened against the peptide, and then were screened against CGEN-R1, produced in bacteria and purified according to the IMAC process above (with nickel beads). The screening process was performed with an ELISA. Briefly, the procedure was as follows. A solution of peptide (10 μg/ml, SEQ ID NO:27) in 100 mM carbonate buffer pH 9.6 was prepared, of which 100 μl/well was added to each well of peptide immobilizer microplates (Exiqon). The plates were incubated overnight at 4° C. under gentle shaking. The plates were then washed with PBST (0.05% Tween). Antibody-containing serum was diluted in PBST (100 μl/well of a solution of 1:1000 into PBST) at the desired concentration, and incubated for 1 h at R.T (room temperature) under gentle shaking. The plates were again washed with PBST and incubated with 100 μl/well goat-anti-rabbit Ig/HRP (Jackson #111-035-144 lot 58940), diluted into PBST solution (1:50,000 from glycerol stock) for 1 hour at R.T. under gentle shaking.

The plates were again washed with PBST and incubated with 100 μl of mixture of solution I and solution 2 of TMB (from Bender # BMSSSL01 and BMSSSL02 at 1:1), followed by color development for 10 min at R.T. The reaction was stopped with H₂SO₄ 4N (4 ml sulfuric acid diluted in 32 ml H₂O, 100 μl/well), after which the optical density was measured at 450 nm.

As an example, the results for bleed 4 were as follows (Table II):

TABLE II
screening of polyclonal antibodies in test bleed 4
against the immunizing peptide (SEQ ID NO: 27):

| Rabbit | Dilution (1/x) | OD |
|---|---|---|
| 1561 | 3000 | 1.4435 |
| | 9000 | 0.414 |
| | 27000 | 0.0345 |
| | 81000 | −0.016 |
| 1562 | 3000 | 3.153 |
| | 9000 | 2.463 |
| | 27000 | 1.2585 |
| | 81000 | 0.2745 |

Later bleeds provided stronger ELISA results (not shown).

The antibody was then purified from rabbit 1562 by ammonium sulfate precipitation. Briefly, a saturated solution of ammonium sulfate was prepared by adding 380 gr to 500 ml water and boiling the solution. The serum was thawed and centrifuged at 10,000 rpm, 4° C. for 5 min. 1 vol PBS was added to each vol serum, and stirred at 4° C.

1 volume of saturated ammonium sulfate was then added under stirring for at least 2 hrs on ice. The solution was centrifuged 15 min at 10000 rpm at 4° C. to precipitate IgG. The pellet was resuspended in 5 ml PBS and dialyzed overnight at 4° C. against PBS+0.05% azide. The precipitated serum was filtered with a 0.45 μm filter.

Affinity purification was then performed with the peptide according to SEQ ID NO:27, in an immunoaffinity column, linked to sulfolink beads (Pierce # 20401). The column was prepared according to manufacturer's instructions. The serum to be purified was mixed with sulfolink beads and incubated under gentle shaking (1 hr at R.T. and 2 hrs at 4° C.), after which the beads were packed into a column.

The column was washed with TRIS 100 mM, followed by binding buffer containing 0.5M NaCl. The IgG was eluted by applying elution buffer: 0.1M Glycine pH3 (fraction size: 0.5 ml), followed by phosphate buffer 100 mM pH 111 to elute another fraction of IgG. The antibodies were then frozen for storage. Western blots showed a highly purified antibody (results not shown).

SDS-PAGE of CGEN-R1 and IL-1Ra WT with Immunoblot Analysis

Purified and crude preparations of the wild type and splice variant proteins (WT and CGEN-R1, respectively) were resuspended in 200 μl 1×SDS-sample buffer containing 50 mM DTT (crude preparation). Following boiling for 5 min and subsequent 5 min centrifugation, samples were loaded on Nu-PAGE gel buffer system (In-Vitrogen).

Following electrophoresis, for performing Western blots, gels were washed with cold transfer buffer for 15 min and taken for transfer to Nitrocellulose membranes for 60 min at 30 V using In-Vitrogen's transfer buffer and X-Cell II blot module. Following transfer, blots were blocked with PBS-1% fat milk-Tween-20 (0.3% protein, 0.04% Tween-20) for at least 60 min at room temperature or overnight at 4° C. Following blocking, blots were incubated with antibodies (either the previously described anti-splice variant antibodies or a commercially available anti-wild type IL-1Ra antibody) at ˜1 μg/ml for 1-3 hrs, washed with block solution, incubated with respective peroxidase-conjugated antibodies, washed with PBS-Tween-20 solution, followed by ECL.

Other gels underwent Coomassie staining or silver staining as described below.

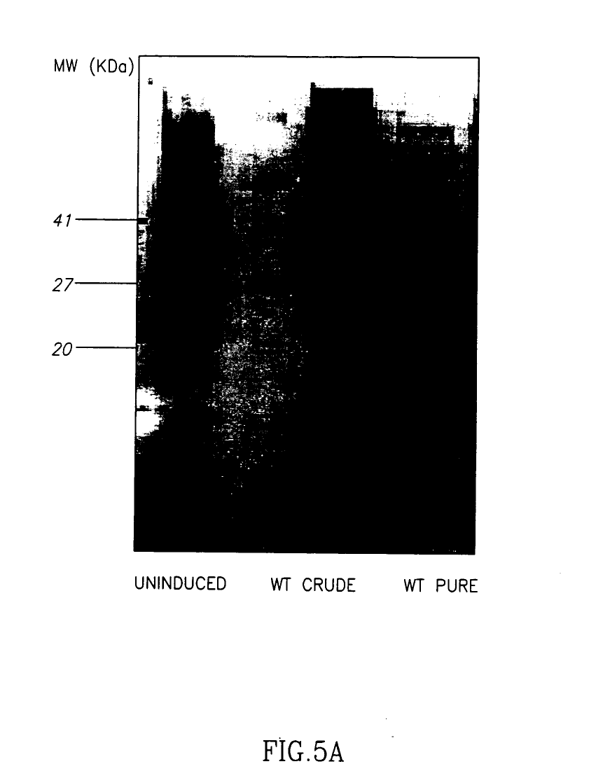

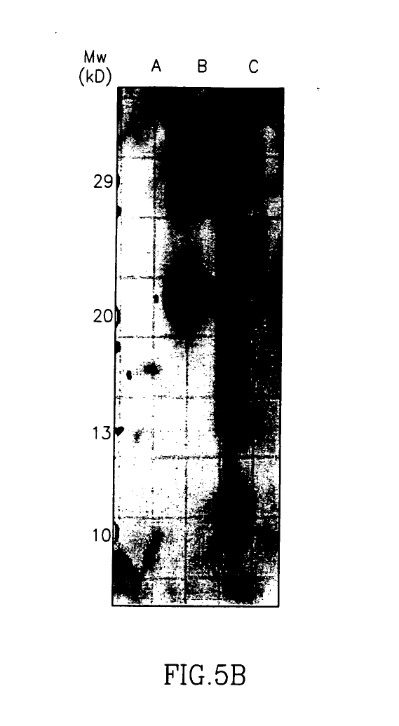

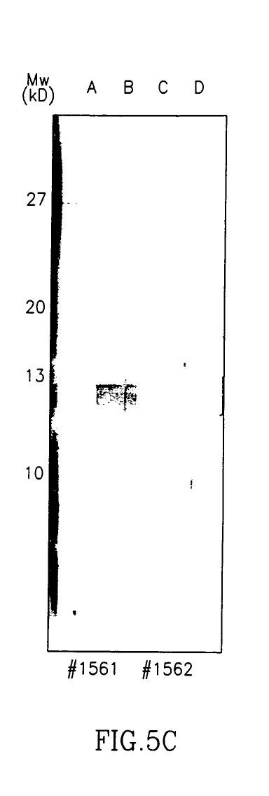

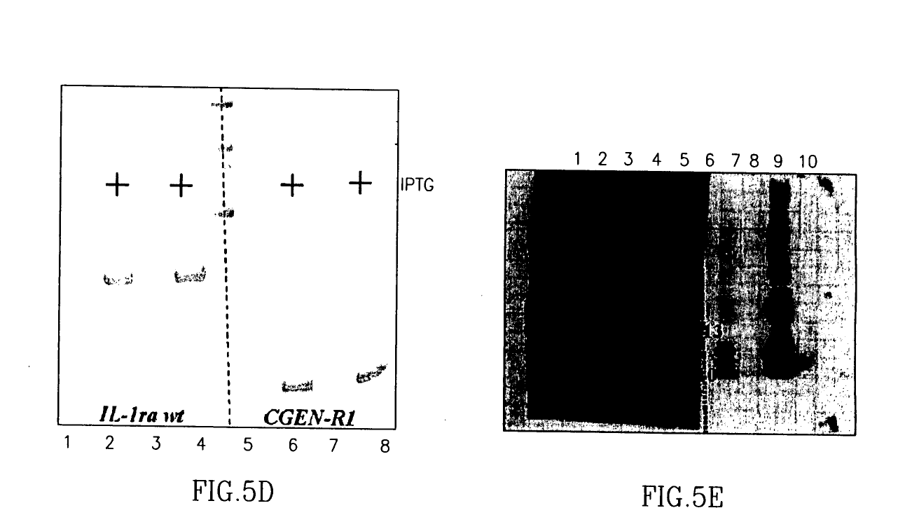

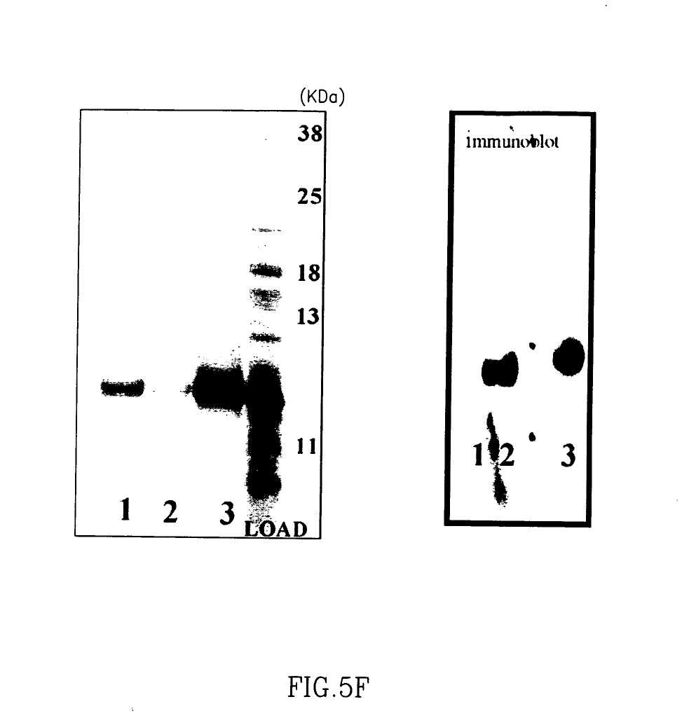

Results of gel electrophoresis with Coomassie staining alone are shown in FIG. 5A for the wild type protein; it should be noted that maximal expression of wild type IL-1Ra protein was induced by 0.1 mM IPTG and an incubation time of 3 hrs. Similarly, maximal expression was induced for the splice variant CGEN-R1 by using 0.1 mM IPTG and 3 hrs incubation time.

FIG. 5B shows that a commercially available anti-wild type IL-1Ra polyclonal antibody (rabbit polyclonal, code P-3001, Endogen lot number EA61243, available from Almog Diagnostics, Shoham, Israel) recognized both the commercial IL-1Ra (R&D, ˜19 kD, lane A) and the IL-1Ra WT protein (˜22 kDa, lane B). The difference in size is probably due to the additional 22 amino acid plasmid-encoding domain at the N-terminus of the Gateway constructs. The polyclonal Ab also appeared to recognize CGEN-R1 at ˜10.7 kDa (lane C).

FIG. 5C depicts the reactivity of pre-immune (lanes A and C) and hyperimmune sera (lanes B and D) to CGEN-R1 following immunization and boosts of 2 separate rabbits (#1561 and #1562, described above). The expected size of the CGEN-R1 variant is 8.2 kDa plus 2.5 kDa of the His-tag (FIG. 5B).

FIG. 5D shows Coomassie staining of WT IL-1Ra and CGEN-R1 proteins purified by IMAC from the lysates of induced cultures. Lanes 2 and 6 represent expression 2 hrs following IPTG addition. Lanes 4 and 8 shows expression following 3 hrs IPTG addition. The other lanes (1, 3, 5, 7) are lysates of the respective uninduced cultures).

FIG. 5E shows a Western blot with protein after IMAC purification that was performed with polyclonal 1561 anti-CGEN-R1 rabbit sera, first bleed (right); and the previously described commercial polyclonal antibody [Endogen] (left). Lanes 1, 3, 7 and 9 represent purified CGEN-R1 from native extracts under native conditions; lanes 2 and 8 represent crude extracts of CGEN-R1 under denaturing conditions; lanes 4 and 10 represent the insoluble fraction (inclusion bodies) left after native extraction; and lane 5 represents 75 ng of commercial IL-1Ra. CGEN-R1 was detected in lanes 7 and 9 with anti-CGEN-R1 bleeds, and weakly with polyclonal anti-IL-1Ra.

Following purification of CGEN-R1 by Prospec Tany TechnoGene Ltd (Rehovot Israel), the protein was electrophoresed as previously described. The gel was then either silver stained (FIG. 5F, left panel) or a Western blot was performed with the previously described anti-CGEN-R1 sera (FIG. 5F, right panel). For the silver stained gel, the “LOAD” lane shows initial load material following solubilization, while lanes 1-3 show different amounts of final re-folded, solubilized material. For the Western blot, the lanes are as follows: 1: IL-1 RA WT (pure); 2: CGEN-R1 (pure); 3: CGEN-R1 (total lysate).

### Example 5

### Functional Assays of IL-1 Receptor Antagonists

Inhibition of IL-8 secretion

IL-1β-induced secretion of IL-8 in T-24 cells (based on a paper by Schwartz et al, 1999, showing IL-8 secretion in A549 cells) was used to examine the functionality of CGEN-R1. The assay was carried out as follows: T24 cells were plated in 6-well plates at 2×10⁵ cells/well. After 24 hrs, cells were washed twice with PBS and changed with medium without serum. After 24 hrs, IL-1β (75 μg/ml) was added to cells with or without IL-1Ra or CGEN-R1. Each experiment included a negative control without IL-1β. Each test point was tested in duplicate wells. After 24 hrs, supernatants were collected, centrifuged for 5 min at 2000 rpm, and kept at −70° C. Secretion of IL-8 in the supernatants was measured by a commercial human ELISA kit. The secretion of IL-8 was high (6000 μg/ml) and required diluting the supernatants before the ELISA test, at a 1:30 dilution.

The activity of bacterial CGEN-R1, purified by native buffer (NLB) or 2M urea extraction, was tested. Several mock samples were used: 1) “Wash 2”—the second wash fraction after binding of CGEN-R1 extract to Ni-beads (see IMAC purification process description), this sample contains 10 mM imidazole which elutes proteins that are weakly bound to the beads (NB # 112 p137). The logic of using this sample was to detect whether there is an unspecific biological activity in the proteins that bind to the beads. 2) “old mock”—same bacterial host/no plasmid/no antibiotics.

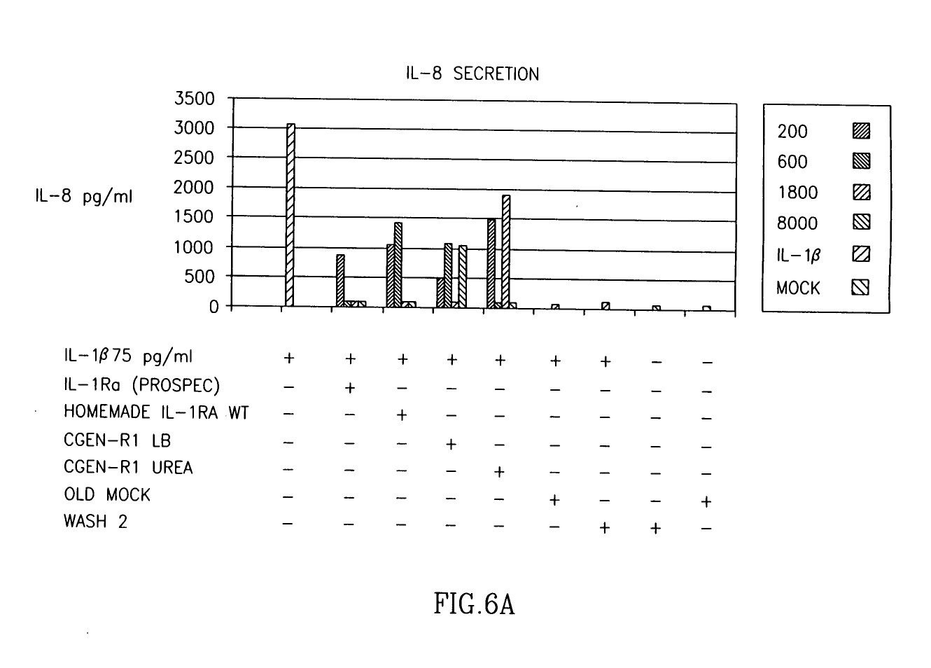

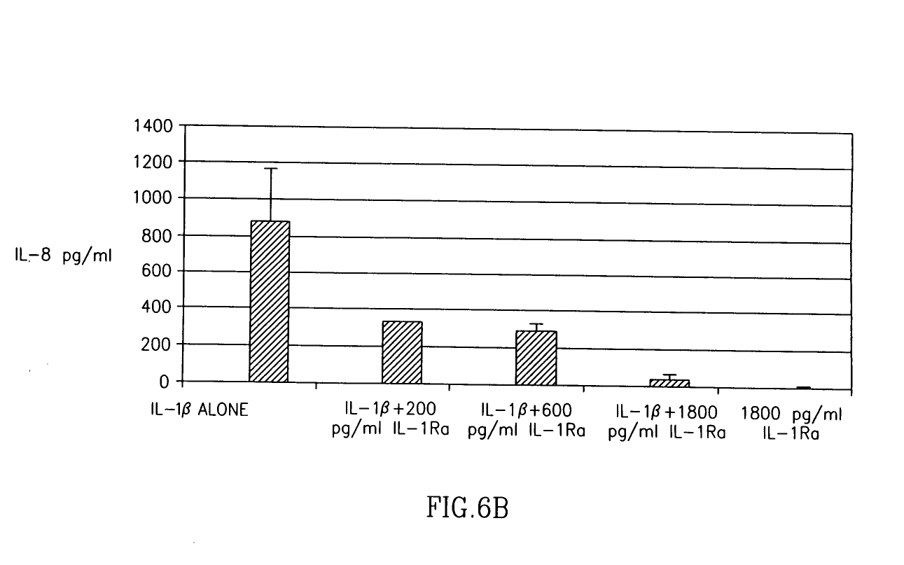

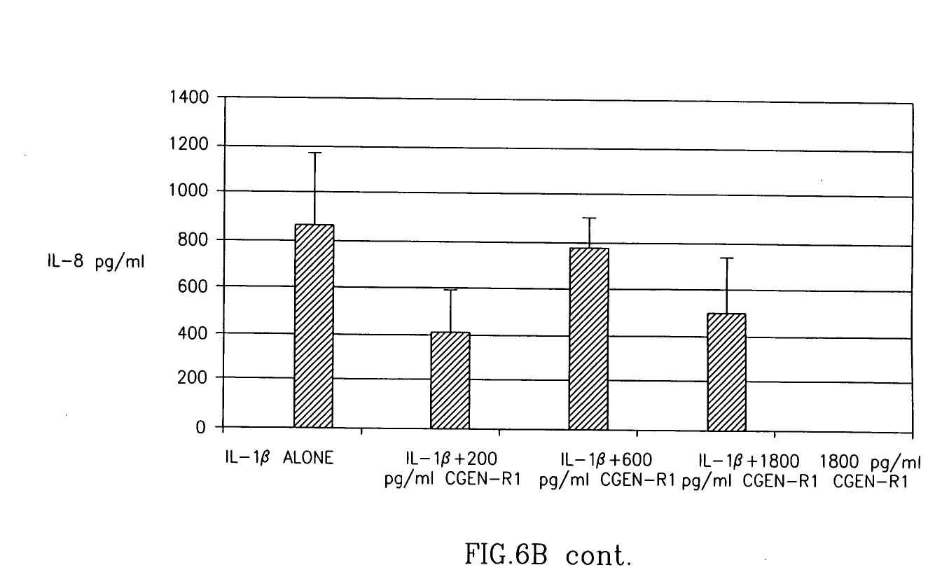

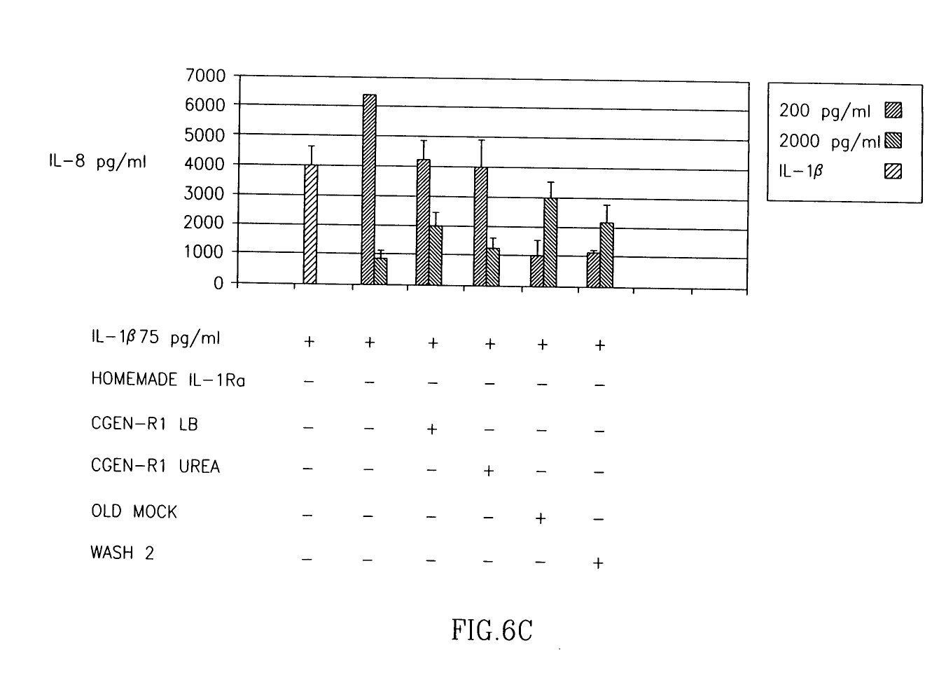

The results in FIG. 6A show that the commercial IL-1Ra WT (obtained from Prospec Tany TechnoGene Ltd (Rehovot Israel)) inhibited the IL-8 secretion over 70% at 200 μg/ml, with 100% inhibition at 600 μg/ml or above. IL-1Ra WT prepared by the present inventors (homemade) showed a similar degree of inhibition (70% at 200 μg/ml, 50% at 600 μg/ml and 100% at 1800 and 8000 μg/ml). CGEN-R1 protein that was purified with either 2M urea (CGEN-R1 urea) or native lysis buffer (CGEN-R1 LB) inhibited IL-8 secretion to at least some extent. As can be seen, the mock samples (old mock and wash 2) also have a non-specific inhibitory effect on IL-8 secretion; however, CGEN-R1 and the wild type IL-1Ra both showed greater specific (dose-dependent) inhibitory activity.

These results are supported by FIG. 6B, which shows that both IL-1Ra WT and CGEN-R1 protein were able to inhibit IL-1β-stimulated IL-8 secretion from T24 cells. This experiment compared the inhibitory effects of IL-1Ra WT (200-1800 μg/ml; Prospec Tany TechnoGene Ltd (Rehovot Israel)) and re-folded CGEN-R1 (200-1800 μg/ml, Prospec Tany TechnoGene Ltd (Rehovot Israel)) on IL-1β-stimulated IL-8 production. From these data, IL-1β caused a consistently reliable IL-8 level of secretion, which was suppressed in a dose-response manner by IL-1Ra WT. In the presence of CGEN-R1, an inhibitory response was observed.

FIG. 6C also shows IL-1β-stimulated IL-8 secretion from T24 cells. This secretion was inhibited in a dose-dependent manner by IL-1Ra purchased from Prospec or prepared by the present inventors, and was also inhibited in a dose-dependent manner by CGEN-R1 protein that was purified with either 2M urea (CGEN-R1 urea) or native lysis buffer (CGEN-R1 LB). However, both of the negative controls showed a reverse dose-dependency, indicating the presence of non-specific inhibitors and also potentially agonists in the crude cell medium and/or additional column wash preparations.

### Example 6

### Expression of CGEN-R1 in HepG2 Cell Line

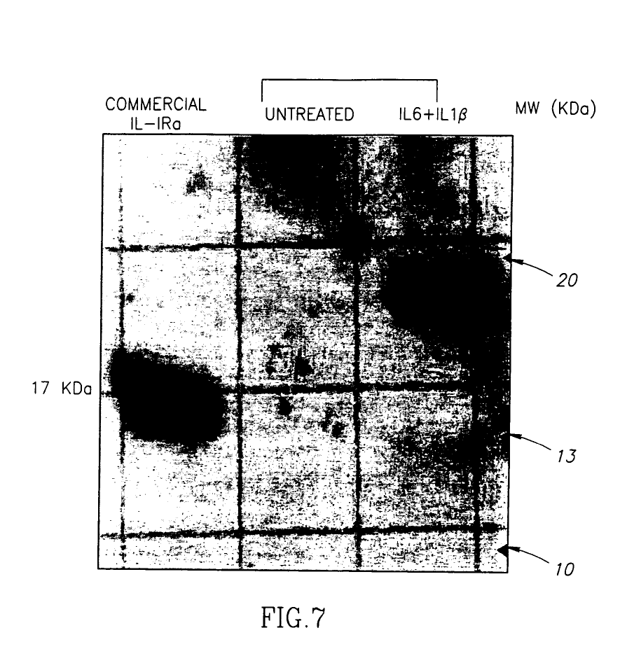

IL-1β (1 ng/ml) and IL-6 (10 ng/ml) were added to conditioned medium of HepG2 cells, and proteins secreted to the medium were isolated, separated by SDS-PAGE and Western Blotted. Polyclonal rabbit antibody, anti hrIL-1Ra, was used for the detection of secreted IL-1Ra (Endogen, described above). Significant amounts of IL-1Ra were secreted from cells treated with IL-1β and IL-6, whereas no detectable amounts were found in the medium of untreated cells in a conditioned medium. The anti hrIL-1Ra antibody could also recognize a commercial IL-1Ra protein. In the proteins extracted from treated, conditioned medium, a faint band of about 13 kDa could be also detected. This result suggests that the new splice variant CGEN-R1 is also produced and secreted from the cells treated with IL-1β and IL-6 (FIG. 7).

### Example 7

### Pharmaceutical Compositions and Treatment

As described previously, the CGEN-R1 splice variant of IL-1Ra according to the present invention (and/or other agents according to the present invention as described previously) may optionally be used in pharmaceutical compositions and/or for the treatment of a number of different diseases and/or pathological conditions. Examples of such diseases or conditions include but are not limited to, rheumatoid arthritis, asthma, inflammation in general, localized inflammation (optionally and preferably inflammation of a joint, for example in synovial tissue), inflammatory bowel disease, transplant rejection, bone marrow transplantation, cancers (including but not limited to leukemias and myelomas), arteriosclerosis, Alzheimer's disease, septic shock, graft versus host disease, reducing, ameliorating or eliminating reperfusion injury, and localized treatment of inflamed mucosal tissue lining a cavity (such as with the ear, nose or sinus for example). Generally, such diseases or conditions include but are not limited to diseases or conditions requiring IL-1 induced expression of a leukocyte adhesion molecule by endothelial cells, or resulting from IL-1 induced adhesion of leukocytes to endothelial cells.

According to other preferred embodiments of the present invention, CGEN-R1 and/or other agents of the present invention as described below may optionally be used for treatment of acute pancreatitis; ALS; cachexia/anorexia; asthma; atherosclerosis; chronic fatigue syndrome; diabetes (e.g., insulin diabetes); glomerulonephritis; graft versus host rejection; hemorrhagic shock; hyperalgesia, inflammatory bowel disease; inflammatory conditions of a joint including osteoarthritis, psoriatic arthritis and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS); multiple myeloma; multiple sclerosis; myelogenous (e.g., AML and CML) and other leukemias; myopathies (e.g., muscle protein metabolism, specifically in sepsis); osteoporosis; Parkinson's disease; chronic pain; pre-term labor; psoriasis; reperfusion injury; septic shock; side effects from radiation therapy, temporal mandibular joint disease, tumor metastasis; or an inflammatory condition resulting from strain, sprain, cartilage damage, trauma, orthopedic surgery, or infection The agents of the present invention can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect (an agent according to the present invention).

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections. Alternatively, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated-sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that treatment of cancer according to the present invention may be combined with other treatment methods known in the art (i.e., combination therapy). Thus, treatment of cancer may be combined with, for example, radiation therapy, antibody therapy and/or chemotherapy.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is to be understood that the phraseology or terminology employed herein is for the purpose of description and not of limitation. The means, materials, and steps for carrying out various disclosed chemical structures and functions may take a variety of alternative forms without departing from the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An isolated nucleic acid comprising a polynucleotide sequence encoding a polypeptide capable of binding to a mammalian IL-1 receptor which is at least 80% homologous to a polypeptide comprising an amino acid sequence selected from the group consisting of SEQ ID NO:2, SEQ ID NO:4 and fragments, variants and analogs thereof.

2. The isolated nucleic acid according to claim 1 wherein the polynucleotide sequence encodes a polypeptide which is at least 85% homologous to a polypeptide comprising an amino acid sequence selected from the group consisting of SEQ ID NO:2, SEQ ID NO:4 and fragments, variants and analogs thereof.

3. The isolated nucleic acid according to claim 1 wherein the polynucleotide sequence encodes a polypeptide which is at least 90% homologous to a polypeptide comprising an amino acid sequence selected from the group consisting of SEQ ID NO:2, SEQ ID NO:4 and fragments, variants and analogs thereof.

4. The isolated nucleic acid according to claim 1 wherein the polynucleotide sequence encodes a polypeptide which is at least 95% homologous to a polypeptide comprising an amino acid sequence selected from the group consisting of SEQ ID NO:2, SEQ ID NO:4 and fragments, variants and analogs thereof.

5. The isolated nucleic acid according to claim 1 wherein the polynucleotide sequence encodes a polypeptide which is 100% homologous to a polypeptide comprising an amino acid sequence selected from the group consisting of SEQ ID NO:2, SEQ ID NO:4 and fragments, variants and analogs thereof.

6. The isolated nucleic acid according to claim 1 wherein the polynucleotide sequence encodes a polypeptide comprising contiguous amino acids having at least 80% homology to positions 44 to 73 set forth in SEQ D NO:4.

7. The isolated nucleic acid according to claim 1 wherein the polynucleotide sequence encodes a polypeptide comprising contiguous amino acids having at least 90% homology to positions 44 to 73 set forth in SEQ ID NO:4.

8. The isolated nucleic acid according to claim 1 wherein the polynucleotide sequence encodes a polypeptide comprising contiguous amino acids having at least 95% homology to positions 44 to 73 set forth in SEQ ID NO:4.

9. The isolated nucleic acid according to claim 1, wherein the polynucleotide sequence encodes a polypeptide comprising contiguous amino acids having 100% identity to positions 44 to 73 set forth in SEQ ID NO:4.

10. An isolated nucleic acid comprising a polynucleotide sequence complementary to the polynucleotide sequence according to claim 1.

11. An isolated nucleic acid comprising a polynucleotide sequence that hybridizes under stringent conditions to a polynucleotide sequence according to claim 1.

12. An isolated nucleic acid comprising a polynucleotide sequence having a nucleotide sequence selected from the group consisting of SEQ ID NO:1, SEQ ID NO:3, SEQ ID NO:5 and SEQ ID NO:6.

13. An isolated nucleic acid comprising a polynucleotide sequence complementary to the polynucleotide sequence according to claim 12.

14. The polynucleotide of claim 1 wherein the polypeptide encoded is devoid of at least one IL-1 activity.

15. The polynucleotide of claim 1 wherein the polypeptide encoded is substantially devoid of IL-1 activity.

16. A polypeptide comprising an amino acid sequence selected from the group consisting of SEQ ID NO:2, SEQ ID NO:4 and fragments, variants and analogs thereof, wherein said polypeptide binds to a mammalian IL-1 receptor.

17. A polypeptide which is at least 80% homologous to the polypeptide of claim 16.

18. A polypeptide which is at least 85% homologous to the polypeptide of claim 16.

19. A polypeptide which is at least 90% homologous to the polypeptide of claim 16.

20. A polypeptide which is at least 95% homologous to the polypeptide of claim 16.

21. The polypeptide according to claim 16 having an amino acid sequence selected from the group consisting of SEQ ID NO:2 and SEQ ID NO:4.

22. The polypeptide according to claim 16 comprising contiguous amino acids having at least 80% homology to positions 44 to 73 set forth in SEQ ID NO:4.

23. The polypeptide according to claim 16 comprising contiguous amino acids having at least 90% homology to positions 44 to 73 set forth in SEQ ID NO:4.

24. The polypeptide according to claim 16 comprising contiguous amino acids having at least 95% homology to positions 44 to 73 set forth in SEQ ID NO:4.

25. The polypeptide according to claim 16 comprising contiguous amino acids having 100% identity to positions 44 to 73 set forth in SEQ ID NO:4.

26. The polypeptide according to claim 16 lacking at least one IL-1 activity.

27. The polypeptide according to claim 16 being substantially devoid of IL-1 activity.

28. A CGEN-R1 protein, comprising a first portion having an amino acid sequence being at least about 90% homologous, and preferably at least about 95% homologous, to amino acids 1-68 of wild type IL-1Ra (SEQ ID NO:8), which are also amino acids 1-68 of SEQ ID NO:2; and a second portion having an amino acid sequence being at least about 80% homologous to an amino acid sequence GEWLPGKPMYVGITSLCPSVCSSMACLHKP (amino acids 69-98 of SEQ ID NO:2), wherein said first and second portions are contiguous and in sequential order.

29. The CGEN-R1 protein according to claim 28, wherein the second portion is at least about 85% homologous to amino acids 69-98 set forth in SEQ ID NO:2.

30. The CGEN-R1 protein according to claim 28, wherein the second portion is at least about 90% homologous to amino acids 69-98 set forth in SEQ ID NO:2.

31. The CGEN-R1 protein according to claim 28, wherein the second portion is at least about 95% homologous to amino acids 69-98 set forth in SEQ ID NO:2.

32. A CGEN-R1 protein, comprising a first portion having an amino acid sequence being at least about 90% homologous, and preferably at least about 95% homologous, to amino acids 1-43 of SEQ ID NO:4; and a second portion having an amino acid sequence being at least about 80% homologous to an amino acid sequence GEWLPGKPMYVGITSLCPSVCSSMACLHKP (amino acids 44-73 of SEQ ID NO:4), wherein said first and second portions are contiguous and in sequential order.

33. The CGEN-R1 protein according to claim 32, wherein the second portion comprises an amino acid sequence being at least about 85% homologous to amino acids 44-73 of SEQ ID NO:4.

34. The CGEN-R1 protein according to claim 32, wherein the second portion comprises an amino acid sequence being at least about 90% homologous to amino acids 44-73 of SEQ ID NO:4.

35. The CGEN-R1 protein according to claim 32, wherein the second portion comprises an amino acid sequence being at least about 95% homologous to amino acids 44-73 of SEQ ID NO:4.

36. A tail portion of CGEN-R1, which comprises a peptide having an amino acid sequence GEWLPGKPMYVGITSLCPSVCSSMACLHKP (amino acids 69-98 of SEQ ID NO:2), or a sequence at least about 80% homologous to said amino acid sequence.

37. The tail portion of CGEN-R1 according to claim 36, having a sequence at least about 85% homologous to amino acids 69-98 set forth in SEQ ID NO:2.

38. The tail portion of CGEN-R1 according to claim 36, having a sequence at least about 90% homologous to amino acids 69-98 set forth in SEQ ID NO:2.

39. The tail portion of CGEN-R1 according to claim 36, having a sequence at least about 95% homologous to amino acids 69-98 set forth in SEQ ID NO:2.

40. A bridge portion of SEQ ID NO:2, comprising a polypeptide having a length “n”, wherein n is at least about 10 amino acids in length, wherein at least two amino acids of said polypeptide comprise EG, said polyptide having a sequence starting from any of amino acid numbers 68−x to 68 of SEQ ID NO: 2; and ending at any of amino acid numbers 69+((n−2)−x) of SEQ ID NO: 2, wherein x varies from 0 to n−2; and wherein 69+((n−2)−x) cannot be larger than 98.

41. The bridge portion of claim 40, wherein n is at least about 20 amino acids in length.

42. The bridge portion of claim 40, wherein n is at least about 30 amino acids in length.

43. The bridge portion of claim 40, wherein n is at least about 40 amino acids in length.

44. The bridge portion of claim 40, wherein n is at least about 50 amino acids in length.

45. The bridge portion of claim 40, comprising a homologous polypeptide having at least 80% homology to said polypeptide

46. The bridge portion of claim 40, comprising a homologous polypeptide having at least 90% homology to said polypeptide.

47. The bridge portion of claim 40, comprising a homologous polypeptide having at least 95% homology to said polypeptide.

48. A bridge portion of SEQ ID NO:2, comprising a polypeptide having a length “n”, wherein n is about 4 to about 9 amino acids in length, wherein at least two amino acids of said polypeptide comprise EG, having a sequence starting from any of amino acid numbers 68−x to 68 of SEQ ID NO: 2; and ending at any of amino acid numbers 69+((n−2)−x) of SEQ ID NO: 2, wherein x varies from 0 to n−2; and wherein 69+((n−2)−x) cannot be larger than 98.

49. A peptide having the amino acid sequence set forth in SEQ ID NO:27.

50. An antibody which binds to at least one epitope of a polypeptide having an amino acid sequence selected from the group consisting of SEQ ID NO:2, SEQ ID NO:4, and fragments, variants and analogs thereof.

51. An antibody which binds to at least one epitope of a polypeptide having an amino acid sequence according to claim 16.

52. An antibody which binds to at least one epitope of a peptide having the amino acid sequence set forth in SEQ ID NO:27.

53. An antibody-antigen complex formed from an antibody and epitope according to claim 50.

54. An expression vector comprising the polynucleotide sequence according to claim 1.

55. A host cell comprising the vector according to claim 54.

56. A process for producing a polypeptide comprising;
a. culturing the host cell of claim 55 under conditions suitable to produce the polypeptide encoded by said polynucleotide and;
b. recovering said polypeptide.

57. A method for detecting a polynucleotide which encodes IL-Ra in a biological sample comprising the steps of:
a. hybridizing the polynucleotide sequence according to claim 13 to a nucleic acid material of a biological sample, thereby forming a hybridization complex; and
b. detecting the hybridization complex, wherein the presence of the complex correlates with the presence of a polynucleotide encoding IL-1Ra in the biological sample.

58. A pharmaceutical composition comprising as an active ingredient a polynucleotide sequence according to claim 1, and further comprising a pharmaceutically acceptable diluent or carrier.

59. A pharmaceutical composition comprising as an active ingredient an expression vector according to claim 54, and further comprising a pharmaceutically acceptable diluent or carrier.

60. A pharmaceutical composition comprising as an active ingredient a host cell according to claim 55, and further comprising a pharmaceutically acceptable diluent or carrier.

61. A pharmaceutical composition comprising as an active ingredient a polypeptide according to claim 16, and further comprising a pharmaceutically acceptable diluent or carrier.

62. A method for preventing, treating or ameliorating an IL-1 related disease or disorder, comprising administering to a subject in need thereof a pharmaceutical composition comprising as an an active ingredient a polypeptide according to claim 16.

63. A method for preventing, treating or ameliorating an IL-1 related diseases or disorder, comprising administering to a subject in need thereof a pharmaceutical composition according to claim 58.

64. A method according to claim 62 wherein the IL-1 related disease or disorder is selected from the group consisting of acute pancreatitis; ALS; Alzheimer's disease; cachexia/anorexia; asthma; atherosclerosis; chronic fatigue syndrome; diabetes; glomerulonephritis; graft versus host rejection; hemorrhagic shock; hyperalgesia; inflammatory bowel disease; inflammatory conditions of a joint: osteoarthritis, psoriatic arthritis, rheumatoid arthritis; ischemic injury; cerebral ischemia; neurodegeneration; lung diseases; multiple myeloma; multiple sclerosis; myelogenous; leukemia; myopathies: muscle protein metabolism; osteoporosis; Parkinson's disease; chronic pain; pre-term labor; psoriasis; reperfusion injury; septic shock; side effects from radiation therapy; temporal mandibular joint disease; tumor metastasis;
inflammatory condition resulting from strain, sprain, cartilage damage, trauma, orthopedic surgery, and infection.

65. An oligonucleotide sequence of at least 17 bases specifically hybridizable with a polynucleotide sequence according to claim 1, so as to direct exponential amplification of a portion thereof in a nucleic acid amplification reaction.

66. An oligonucleotide having a nucleotide sequence selected from the group consisting of SEQ ID NO:15, SEQ ID NO:16, SEQ ID NO:17, SEQ ID NO:18, SEQ ID NO:19, SEQ ID NO:20, SEQ ID NO:21 and SEQ ID NO:22.

67. An isolated DNA sequence produced by using a pair of primers comprising a nucleotide according to claim 65 in a DNA amplification method, wherein the primers are combined in the assay with the polynucleotide sequence as a template.
